(19) United States
(12) Patent Application Publication
(10) Pub. No.: US 2026/0166196 A1
(43) Pub. Date: Jun. 18, 2026

(54) ROS-RESPONSIVE THERONOSTIC BIOSENSOR

(71) Applicant: University of Manitoba, Winnipeg (CA)

(72) Inventors: Farinaz J. SHARIATZADEH, Winnipeg (CA); Sarvesh LOGSETTY, Winnipeg (CA); Song LIU, Winnipeg (CA)

(21) Appl. No.: 19/409,162

(22) Filed: Dec. 4, 2025

Related U.S. Application Data
(60) Provisional application No. 63/734,306, filed on Dec. 16, 2024.

(51) Int. Cl. A61L 15/26; A61L 15/44; A61L 15/56; C12Q 1/37

(52) U.S. Cl. CPC A61L 15/26; A61L 15/44; A61L 15/56; C12Q 1/37; A61L 2300/406; A61L 2400/12; G01N 2333/918; G01N 2333/93; G01N 2333/96433; G01N 2333/96491

(57) ABSTRACT

The theranostic wound dressing that combines diagnostic functions with therapeutic actions, including the targeted release of antibiotics, which enhances treatment efficiency and reduces the likelihood of prolonged infections or the development of antibiotic resistance. The present invention provides a theranostic wound dressing, that includes citric acid and a modified hemicyanine dye immobilized within polymer nanofibers formed as a nanofibrous matrix, the matrix immobilizing nanoparticles encapsulating at least one antimicrobial compound. The nanoparticles are structurally responsive to reactive oxygen species (ROS) to effectively release an antimicrobial. The hemicyanine dye is adapted to react with a color-change in the presence of pathogenic enzymes. Ester bonds present in the hemicyanine dye are cleavable by pathogenic enzymes secreted by pathogenic bacteria and fungi. Cleaving the ester bonds present in the hemicyanine dye increases intramolecular charge transfer in the hemicyanine dye and induces the color-change.

### CROSS REFERENCE TO RELATED APPLICATIONS

This application claims the benefit of U.S. provisional patent application 63/734,306 filed Dec. 16, 2024 titled “ROS-RESPONSIVE THERONOSTIC BIOSENSOR” assigned to the University of Manitoba as Applicant and named inventors Farinaz J. Shariatzadeh, Sarvesh Logsetty, and Song Liu, and which is expressly incorporated herein by reference in its entirety.

### FIELD OF INVENTION

The present disclosure relates generally to chromatic biosensors for use in diagnostics and wound healing. More particularly, the disclosure relates to chromatic biosensors responsive to enzymatic activity.

All of the references, patents, and patent applications that are referred to herein are incorporated by reference in their entirety as if they had each been set forth herein in full. Note that this application is one in a series of applications by the Applicant covering methods and apparatus for enabling biomedical applications of nanofibers. The term “fiber” and the term “nanofiber” may be used interchangeably, and neither term is limiting. The disclosure herein goes beyond that needed to support the claims of the particular invention set forth herein. This is not to be construed that the inventor is thereby releasing the unclaimed disclosure and subject matter into the public domain. Rather, it is intended that patent applications will be filed to cover all of the subject matter disclosed below. Also, please note that the terms frequently used below “the invention” or “this invention” is not meant to be construed that there is only one invention being discussed. Instead, when the terms “the invention” or “this invention” are used, it is referring to the particular invention being discussed in the paragraph where the term is used.

### BACKGROUND

Wound infection is a global healthcare issue that affects the healing process. Appropriate wound dressing material can reduce the risk of infection by reducing or eliminating the invasion of pathogens. The use of antibacterial materials or agents in wound dressings can reduce risk of infection. Early detection of bacterial presence at low thresholds enables early intervention to prevent worsening of infection and the complications and negative outcomes that often result.

Wound management is an essential aspect of clinical care, primarily due to the complications that arise from infections, which can significantly hinder the healing process and increase the likelihood of severe scarring. Infections at wound sites typically trigger an inflammatory response that can delay tissue repair and promote excessive collagen deposition, ultimately resulting in pronounced and extensive scar formation. The emergence of drug-resistant bacteria, such as methicillin-resistant Staphylococcus aureus (MRSA), poses a challenge in treating bacterial infections, contributing to elevated mortality rates and a higher incidence of resistant strains. In 2019, antibiotic resistance was linked to approximately 4.95 million deaths globally, with 1.27 million directly attributable to drug-resistant infections. This number is projected to rise to 10 million annually by 2050. In the United States alone, drug-resistant bacteria infect at least 2 million people each year, resulting in at least 23,000 deaths. The misuse and overuse of antibiotics are significant contributors to the proliferation of these resistant bacteria. This growing concern underscores the urgent need for advanced therapeutic strategies, such as the development of smart wound dressings capable of detecting bacterial infections early and delivering targeted treatments effectively, thereby mitigating the risk of developing drug-resistant infections.

Theranostic wound dressings represent a significant advancement in wound care by combining therapeutic and diagnostic functions. These innovative dressings are designed to monitor wound conditions and deliver targeted treatments, which can significantly reduce the incidence of wound infections and improve healing outcomes.

Smart and responsive wound dressings are designed to respond to environmental changes within the wound site, such as pH and temperature changes or the presence of specific biochemical markers like lipases and reactive oxygen species (ROS). These environmental biomarkers can facilitate both detection and treatment.

Reactive oxygen species (ROS) are highly reactive forms of molecular oxygen, including the superoxide anion radical, hydrogen peroxide, singlet oxygen, and hydroxyl radical. ROS are generally produced during normal metabolism of oxygen inside the mitochondrial matrix, which acts as their primary source. Basal levels of ROS serve as physiological regulators of normal cell multiplication and differentiation. If the balance of ROS increases more than the scavenging capacity of the intracellular antioxidant system, the cell undergoes a state of oxidative stress with significant impairment of cellular structures. Excessive levels of ROS, for example, can cause severe damage to DNA and proteins.

Oxidative stress especially targets mitochondria, resulting in the loss of mitochondrial membrane potential and initiating mitochondria-mediated apoptosis. Oxidative stress can also lead to the auto-oxidation of sterols, thereby affecting the cholesterol biosynthetic pathway-mainly the post lanosterol derivatives. The intracellular accumulation of oxysterols directs the cell to its autophagic fate and may also induce it to differentiate. ROS, in fact, can play contrasting roles: they can initiate autophagic cell death and function as a survival mechanism through induction of cytoprotective autophagy in several types of cancer cells.

The integration of biosensing capabilities within dual-responsive wound dressings is crucial for real-time detection of infections, especially at low bacterial concentrations that traditional methods might not promptly identify. Such dressings also allow for immediate therapeutic responses, potentially preventing severe complications associated with wound infections. This responsiveness ensures that therapeutic agents, such as antibacterial drugs, are released precisely when pathogens are present, thereby maximizing efficacy and minimizing side effects.

While most theranostic wound dressings developed to date have primarily utilized fluorescent probes or pH indicators for infection detection, these methods generally require additional equipment and do not exclusively indicate infection, thus limiting their clinical utility. Moreover, several responsive dressings have been designed to release drugs upon external stimuli such as light or magnetic fields. For instance, Ran P, et al. engineered a theragnostic injectable hydrogel that detects infections through pH changes and combats bacteria after laser illumination, generating ROS that not only acts as a crosslinking agent for hydrogel formation but also possesses antibacterial properties (Ran P, Xia T, Zheng H, Lei F, Zhang Z, Wei J, et al. Light-triggered theranostic hydrogels for real-time imaging and on-demand photodynamic therapy of skin abscesses. Acta Biomater 2023; 155:292-303). Similarly, Yang J, et al. utilized photodynamic technology for in situ bacterial detection and eradication using a dressing made from carboxymethyl chitosan and oxidized sodium alginate, with added 4-methylumphulone beta-D-glucoside (MUG) nanoparticles coated with titanium dioxide (Yang J, He Y, Li Z, Yang X, Gao Y, Chen M, et al. Intelligent wound dressing for simultaneous in situ detection and elimination of pathogenic bacteria. Acta Biomater 2024; 174:177-90). Upon detection of bacteria, MUG interacts with pathogenic enzymes to produce 4-methylumbellione, which emits blue fluorescence. After detection, near-infrared irradiation can be used to activate the nanoparticles to regenerate ROS, which serves as the antibacterial agent. Although both dressings show promising detection capabilities and antibacterial properties, their reliance on additional light sources for detection and activation limits their broader application, particularly in resource-limited environments.

Addressing the limitations of external radiation units, Pang Q, et al. developed a bilayer wound dressing (Pang Q, Lou D, Li S, Wang G, Qiao B, Dong S, et al. Smart Flexible Electronics-Integrated Wound Dressing for Real-Time Monitoring and On-Demand Treatment of Infected Wounds. Advanced Science 2020; 7). The top layer comprises PDMS integrated with flexible electronics for temperature sensing and diodes for UV light emission, while the bottom layer features a hydrogel responsive to UV light with UV-cleavable linkages between gentamicin and polyethylene glycol. Upon detecting an infection-induced temperature rise (above 40° C.), data is transmitted to a smartphone via Bluetooth, triggering an alert. The software includes a switch to activate the UV diodes in the top layer, subsequently releasing the antibiotic from the bottom layer through UV-induced degradation. This system eliminates the need for an external light source; however, its dependence on smartphone connectivity and temperature fluctuations could pose challenges in wound care management.

Given the limitations of existing theranostic wound dressings, there is a pressing need for an innovative solution that utilizes bacterial by-products for detection and wound environmental biomarkers for targeted drug release.

### SUMMARY

Pathogenic bacteria and fungi secrete various enzymes that help them invade host tissues and evade the immune system. Some key enzymes include the following:

### Pathogenic Bacteria:

1. Proteases: Break down proteins in host tissues, aiding in invasion and nutrient acquisition.
2. Lipases: Degrade lipids, helping bacteria to penetrate fatty tissues.
3. Hyaluronidase: Breaks down hyaluronic acid in connective tissues, facilitating the spread of bacteria.
4. Collagenase: Degrades collagen, a major component of connective tissues, aiding in tissue invasion.
5. Coagulase: Causes blood clotting, which can protect bacteria from immune cells.

### Pathogenic Fungi:

1. Pectinases: Degrade pectin, a component of plant cell walls, facilitating tissue penetration.
2. Proteases: Similar to bacterial proteases, these break down proteins in host tissues.
3. Lipases: Help fungi to degrade lipids and invade fatty tissues.
4. Chitinases: Break down chitin, a component of fungal cell walls, which can help in remodeling fungal structures during infection.

These enzymes play crucial roles in the pathogenicity of bacteria and fungi, enabling them to infect and cause disease in their host. Dyes responsive to these enzymes can be used to indicate though a color-change the presence of pathogens in a wound or on human skin.

The present invention in a preferred embodiment provides a wound dressing that detects bacterial presence in situ via the secretion of lipases, causing a color shift in a nanofiber membrane from yellow to green. This colorimetric change is facilitated by a modified hemicyanine dye incorporated into the shell of core-shell nanofibers composed of polyurethane and polypyrrolidone. In a preferred embodiment, the dye, linked to a fatty acid through an ester bond, undergoes a reaction in the presence of pathogenic lipase; the cleavage of the ester bond enhances intramolecular charge transfer, thus causing the color change. This mechanism allows for the detection of bacteria without external equipment, simplifying usage and reducing system complexity.

The chemical structure of the modified hemicyanine dye in a preferred embodiment is as follows: 1H NMR (CDCl3, 300 MHz) δ 7.65-7.72 (m, 3H), 7.27 (d, J=8.6 Hz, 2H), 7.02 (d, J=16.5 Hz, 1H), 2.62 (t, J=7.6 Hz, 2H), 1.84 (s, 8H), 1.41-1.46 (m, 4H), 0.97 (t, J=7.05 Hz, 3H). 13C NMR (DMSO-d6, 75 MHz): δ 177.5, 175.5, 172.0, 153.8, 146.7, 132.4, 131.3, 123.2, 115.8, 113.1, 112.3, 111.2, 100.1, 55.0, 33.9, 31.0, 25.5, 24.4, 22.3, 14.3. MS (ESI): m/z calcd for C24H23N3O3: 401.1739. Found: 400.1651 [M−1]+. FTIR max (cm-1): 2980-2781 (C—H stretch), 2222 (C N stretch), 1770 (C═O stretch), 1255 (C—O stretch).

Simultaneously, the dressing is designed to release antibiotics in response to ROS, a biomarker significantly elevated in infected wounds as the body's immune response intensifies. In typical human plasma, the concentrations of hydrogen peroxide usually range from 1 to 8 μm, whereas in activated macrophages, these levels can escalate to between 10 and 1000 μm. In a preferred embodiment, the antibiotic ciprofloxacin (CIP) is encapsulated within ROS responsive nanoparticles made from polyethylene glycol b-polypropylene sulfide (mPEG45-b-PPS60), which changes its structure in the presence of ROS, effectively releasing the drug. Use of alternative antimicrobial compounds encapsulated within ROS responsive nanoparticles, including any of clindamycin, doxycycline, minocycline, trimethoprim-sulfamethoxazole (Bactrim), mupirocin (Bactroban), gentamicin, ceftriaxone or analogs thereof, are possible and anticipated.

The responsive delivery system of the present invention not only prevents premature drug release but also addresses potential issues of bacterial resistance and toxicity toward human cells. In a preferred embodiment, incorporation of Citric Acid known for its safety and biocompatibility supports wound healing by maintaining an acidic environment. Besides Citric Acid, several other organic acids are also conducive to wound healing and can be used, including Ascorbic Acid (Vitamin C), Hyaluronic Acid, Glycolic Acid, and Malic Acid.

By integrating both diagnostic and therapeutic functions that operate based on a wound's biological environment, this approach provides a streamlined, cost-effective solution enhancing clinical outcomes through timely and targeted treatment, aligned with the goals of precision medicine in wound care, combining identification of infection and therapeutic action.

In one aspect, the present invention provides a theranostic wound dressing, comprising organic acid and a hemicyanine dye immobilized within polymer nanofibers formed as a nanofibrous matrix.

In another aspect, a core-shell nanofiber has a colorimetric probe incorporated in the shell, wherein the colorimetric probe changes color in the presence of pathogens at a tunable specific threshold.

In another aspect, nanoparticles are immobilized within the nanofibrous matrix, and at least one antimicrobial compound is encapsulated within the nanoparticles.

In another aspect, the nanoparticles comprise polyethylene glycol b-polypropylene sulfide (mPEG45-b-PPS60), which changes structure in the presence of ROS, and allows release of the antimicrobial compound.

In another aspect, the nanoparticles are structurally responsive to reactive oxygen species (ROS).

In another aspect, ester bonds present in the hemicyanine dye are cleavable by enzymes, which increases intramolecular charge transfer in the hemicyanine dye and induces the color-change.

In another aspect, ester bonds present in the hemicyanine dye are cleavable by enzymes secreted by pathogenic fungi, including pectinases, proteases, lipases, and chitinases.

In another aspect, ester bonds present in the hemicyanine dye are cleavable by enzymes secreted by pathogenic bacteria, including proteases, lipases, hyaluronidase, collagenase, and coagulase.

In another aspect, an organic acid incorporated in the nanofibers is selected from any of Citric Acid, Ascorbic Acid (Vitamin C), Hyaluronic Acid, Glycolic Acid, and Malic Acid.

In another aspect, the antimicrobial compound includes any of ciprofloxacin, clindamycin, doxycycline, minocycline, trimethoprim-sulfamethoxazole (Bactrim), mupirocin (Bactroban), gentamicin, ceftriaxone or analogs thereof.

In another aspect, a hydrophobic portion of the mPEG45-b-PPS60 polymer induces self-assembly of the nanoparticles and contributes to their stability.

In another aspect, the nanoparticles comprise ciprofloxacin in a self-assembled polymer structure.

In another aspect, the nanofibers fibers in said nanofibrous matrix comprise citric acid in the range of 3% to 5% and a fiber thickness in the range of 350-450 μm.

In another aspect, the nanofibers fibers in the nanofibrous matrix comprise citric acid in the range of 3.8% to 4.2% and a fiber thickness in the range of 380-420 μm.

In another aspect, the mPEG45-b-PPS60 polymer nanoparticles when freshly assembled exhibit spherical morphology with an average diameter of 213.9=29.5 nm.

In another aspect, the fiber diameters range from 200 nm to 800 nm, with an average diameter of 412±140 nm and the average membrane thickness is 400 μm±20 nm.

In another aspect, the structure of m-PEG₄₄-b-PPS₆₀ is adapted to change in the presence of a ROS agent converting into hydrophilic poly(propylene sulfone).

In another aspect, the present invention provides real-time feedback on infection status within a wound and enables timely intervention, signaling the presence of bacteria and fungi at critical thresholds through a visible color change of the nanofibers comprising a nanofibrous matrix, while sequentially releasing in response to ROS an antimicrobial agent loaded into nanoparticles that are immobilized in the matrix.

In another aspect, the present invention provides healthcare providers, patients, and caregivers with a visual method of monitoring wounds without the need for invasive diagnostics or frequent dressing changes.

In another aspect, the present invention provides a method to combine diagnostic functions with therapeutic actions, such as the targeted release of antibiotics, which enhances treatment efficiency and reduces the likelihood of prolonged infections or the development of antibiotic resistance.

In another aspect, micelles comprise the structure of the theranostic wound dressing providing spherical aggregates of amphipathic molecules with a hydrophobic core and a hydrophilic outer shell comprising a dense PEG-based outer layer imparting a neutral charge, making them non-immunogenic, non-inflammatory, and inhibitory to nonspecific cellular interactions by regulating protein corona formation.

Detecting and treating infections in wound care is increasingly challenging, especially with the rise of antibiotic-resistant bacteria. The present invention provides a nanofibrous theranostic biosensor that delivers both visual infection detection and controlled drug release to combat bacterial resistance. The biosensor incorporates in a preferred embodiment citric acid and ciprofloxacin (CIP)-loaded nanoparticles (NPs) or analogs thereof within a nanofibrous matrix, allowing for a colorimetric response in the presence of bacteria and controlled CIP release upon exposure to reactive oxygen species (ROS), which are elevated in infected wounds. The biosensor's color-changing properties may be evaluated under different conditions. The biosensor is compatible with various wound dressing foam materials and cleaning solutions without interference, and no false positives occur in the presence of salts and proteins. With a detection limit of 1.0E+5 CFU/cm², the biosensor provides vivid visual results within 10 hours depending on pathogen density and type of pathogen triggering the color change. In direct antibacterial tests, 100% bacterial reduction for E. coli (2.9E+10.0±2.6E+9.0 CFU/cm²), MRSA (4.4E+10.0±1.4E+9.0 CFU/cm²), and P. aeruginosa (4.5E+10.0±6.4E+9.0 CFU/cm²) were achieved within 4 hours in the presence of H₂O₂. Citric acid provided untargeted antibacterial action (99.9% for E. coli and 99.999% for MRSA and P. aeruginosa), while ROS-responsive CIP release ensured sustained triggered bacterial elimination (100% and 8 log reduction), significantly enhancing overall efficacy. Cytocompatibility tests confirmed that the biosensor was non-cytotoxic, maintaining over 90% fibroblast viability even after complete drug release. The theranostic biosensor of the present invention effectively prevents untargeted antibiotic release, reducing the risk of bacterial resistance while supporting fibroblast health and proliferation. These results suggest that this biosensor fills a significant unmet need for wound care, offering real-time infection monitoring and targeted antibacterial treatment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows how different aggregate configurations of the dye cause different color changes.

FIG. 2 shows the effect of the addition of different acids to the biosensors and performance impact in AWF solution with and without lipase.

FIG. 3A shows the effect of citric acid concentration and electrospinning time on the color-changing property of the biosensor in the absence of lipase.

FIG. 3B shows the morphology of the biosensor in SEM image and fiber diameter distribution.

FIG. 3C shows a SEM image from the cross-section of the biosensor showing the thickness of the biosensor.

FIG. 4 shows color-changing response of the biosensor in the presence of different buffers at different levels of pH.

FIG. 5 shows color-changing response of the biosensor in the presence of different ions at different concentrations.

FIG. 6 shows color-changing response of the biosensor in the presence of different salts.

FIG. 7 shows color-changing response of the biosensor in the presence of different proteins.

FIG. 8 shows biosensors color changing A) after one hr. in the presence of different lipases from bacteria and fungi sources, and B) during one hr. at different concentrations of L₁.

FIG. 9 shows A) bench test study of biosensors color in the absence (AWF) and the presence (AWF+lipase) of lipase under different lighting settings and at two different angles; B) the color space and color of biosensors in AWF and AWF+lipase; and C) the reflection spectra of AWF and AWF+lipase samples indicate the differences between these two samples.

FIG. 10A shows the performance of the biosensors in the presence of different bacteria, the color changing of the Biosensor in the presence of P. aeruginosa, E. coli, and MRSA under different light sources compared to control with no bacteria, and the flashcard for differentiating infected wounds from non-infected wounds based on the color change.

FIG. 10B shows the color wavelength spectra of biosensors after exposure to the bacteria and control and the color space of the control and bacteria exposed sample.

FIG. 10C shows a flash card developed to help end users differentiate between infected and non-infected wounds.

FIG. 11A shows the compatibility of the fabricated biosensors with different commercially available foam products.

FIG. 11B shows the results of bench testing, where the displayed colors represent the actual biosensor responses.

FIG. 11C shows that among the tested commercially available foams, only Curafoam showed a noticeable color change response within the first 6 hours to all three bacterial strains tested.

FIG. 11D shows color changing responses for biosensors paired with different foam on agar with Simulated Wound Fluid (SWF) in a bacterial test.

FIG. 12 shows the plates after overnight incubation with Curafoam®-paired biosensors and the SEM images of the foam and the biosensors to show the presence of bacteria.

FIG. 13A shows treating the bacteria agar plates with Hypochlorous Acid (HPA) wound cleaning solution either directly pouring the solution or rubbing the agar with gauze soaked in the solution for three different strains of bacteria to evaluate the color change of the biosensors after treatment.

FIG. 13B shows immersing the color-changed biosensors in the HPA solution to evaluate the color stability.

FIG. 13C shows color theory regarding orange and green colors of the dye after cleavage in different environments.

FIG. 14 shows A) sterilized samples with three different dosages; B) the results of the bench tests of the sterilized samples, and C) the results of the bacteria tests for sterilized samples.

FIG. 15 shows A) the foam thickness and penetrated dye through the foam height; and B) the dye stains on top of the foams after removing the biosensors and cross-section of the foams to show the penetrated dye throughout the foams.

FIG. 16 shows A) the cell viability of Human Adult Skin Fibroblast in the presence of three-day extracted biosensors with different dilutions (1-0.001×) compared to only growth media and DMSO, and B) the morphology cells in the presence of extracts.

FIG. 17A shows a schematic of reaction for synthesizing ROS-responsive PEG₄₅-b-PPS₆₀ co-polymer.

FIG. 17B shows FTIR spectra of mPEG, mPEGMA, and mPEG Thioacetate and the NMR spectrum of PEG₄₅-b-PPS₆₀ co-polymer.

FIG. 17C shows NMR data for mPEG45-b-PPS₆₀.

FIG. 18A shows the double-emulsion technique for fabricating ROS-responsive NPs loaded with ciprofloxacin.

FIG. 18B shows self-assembly of PGE₄₅-b-PPS₆₀.

FIG. 19 shows A) TEM image of freshly fabricated NPs; B) the TEM image of NPs after treatment with H₂O₂; and C) the hydrodynamic diameter of fresh NPs and NPs stored in the fridge in PBS for three months.

FIG. 20 shows the morphology of the theranostic biosensor after immersing in NPs solution and drying.

FIG. 21A shows A) the loading efficiency and loading capacity of different concentrations of CIP loaded into NPs, and B) the release profile of CIP from different NPs and NFs loaded with NPs ND.

FIG. 21B shows the PPS segment undergoes a transition from hydrophobic to hydrophilic, where the structure of m-PEG₄₄-b-PPS₆₀ changes in the presence of a ROS agent converting into hydrophilic poly(propylene sulfone).

FIG. 21C shows the release models fitted on the release profile in the presence and absence of ROS agent where disassembly of the nanoparticles triggers the release of the encapsulated drug as illustrated.

FIG. 21D shows NPs and NP/NF systems achieved 100% release within 72 hours, where for NPs alone, more than 50% of the drug was released within 6 hours, and the NP/NF system required 12 hours to reach 60% release.

FIG. 22 shows MIC and MBC of CIP-Loaded ROS-responsive NPs and CIP in the absence and the presence of H₂O₂ as ROS agent.

FIG. 23 shows A) the Zone of Inhibition (ZOI) data for different bacteria for biosensors (NFs), citric acid-containing biosensors (C-NFs), and theranostic biosensors (NPs-C-NFs) having both citric acid and NPs, where all samples were tested in the absence of H₂O₂ and the presence of H₂O₂ in the broth or locally, and B) the ZOI on agars.

FIG. 24A shows direct antibacterial effect of biosensors with different compositions in the presence and absence of H₂O₂.

FIG. 24B shows the time-dependent antibacterial effect of CIP, NPs, and NPs-C-NFs in the presence and absence of H₂O₂ for 5 and 24 hours.

FIG. 25A shows A) IC50 of pure CIP in the absence and presence of H₂O₂, and B) IC50 of CIP-loaded NPs in the absence and presence of H₂O₂.

FIG. 25B shows the Cell viability in the presence of different biosensor extracts at different time points.

FIG. 26 shows log reduction in the number of bacteria and fibroblasts in different conditions in the presence and absence of H2O2: cocktail of antibiotics, No treatment, NFs, NPs, and NFs+NPs.

### DETAILED DESCRIPTION

### Electrospinning Biosensors

The present invention provides electrospun biosensors for detecting pathogens in wounds.

Core-shell PU: PU/PVP/HCy/Citric acid membranes can be fabricated with a co-axial spinneret apparatus (NE300 electrospinner). For core solution of the present invention, 6.0 w/v % PU was dissolved in DMF: THF 1:1 solution overnight at 45.0° C., and for shell solutions, 7.0 w/v % PU, 3.5 w/v % PVP, 4.0% Citric acid and 1.0 w/v % HCy were dissolved in DMF: THF overnight at 45.0° C. The 0.5 v/v % Tween 80 solution was mixed thoroughly with the shell solutions before electrospinning. The electrospinning conditions were maintained at a constant voltage of 20.0 kV, a needle-to-collector distance of 15.0 cm, and pump rates of 0.6 mL/hr for the core solution and 1.0 mL/hr for the shell solution. The morphology of the nanofibers produced was evaluated with a scanning electron microscope at a voltage of 20.0 kV. The membranes were sputtered with gold/palladium prior to scanning.

### Response to Different Solutions

To assess the performance of the biosensors under varying conditions, different buffers, salts, and protein solutions were prepared at various pH levels. The buffers used included artificial wound fluid (AWF, composed of CaCl₂, NaCl, and Tris), phosphate-buffered saline (PBS, containing NaCl, KCl, Na₂HPO₄, and KH₂PO₄), Tris (tris aminomethane), (HOCH₂)₃CNH₂), Tricine (C₆H₁₃NO₅), Bicine (C₆H₁₃NO₄), MES (2-(N-morpholino) ethane sulfonic acid, C₆H₁₃NO₄S), and HEPES (4-(2-hydroxyethyl)-1-piperazineethanesulfonic acid, C₈H₁₈N₂O₄S). These buffers were tested at pH levels of 4.0, 7.0, 8.0, and 11.0. The salt solutions were prepared with specific cations: For sodium (Na⁺), the salts included NaCl, Na₅O₁₀P₃, NaHCO₃, NaH₂PO₄, and Na₂HPO₄. For potassium (K⁺), the salts used were KCl, KI, KH₂PO₄, and K(CH₃COOH). Magnesium (Mg²⁺) was tested using MgCl₂ and Mg (CH₃COOH), while CuBr and FeCl₂ were also included in the tests. Each solution contained a cation concentration of 500 μL. To determine whether ion concentration influenced the biosensor's color-changing properties, Na⁺/Cl⁻, Mg²⁺/Cl⁻, and K⁺/Cl⁻ were tested at concentrations of 5.0, 10.0, 100.0, and 150.0 nM. Additionally, mixtures of two or three of these ions at the same final concentrations were also evaluated.

For protein solutions, bovine serum albumin (BSA, 2%) was added to AWF or solutions containing different salts (NaHCO₃, NaCl, KCl, and CaCl₂)). Fetal bovine serum (FBS) was mixed with AWF (1:1) or peptone water (peptone: NaCl) to investigate further the role of proteins in the biosensors' color-changing properties.

### Response to Different Lipases and Sensitivity of the Biosensor

The color-changing behavior of the biosensors in the presence of different bacterial and fungal lipases was evaluated using a pure water solution. Five different dilutions were prepared for the experiment: 10.0, 5.0, 1.0, 0.5, and 0.1 mg/mL. The lipases used in this study included Amano Lipase PS from Burkholderia cepacia (≥30,000 U/g), Amano Lipase from Pseudomonas fluorescens (≥20,000 U/g), Lipase from Pseudomonas cepacia (30 U/mg), and Lipase from Candida rugosa (≥2 U/mg). All lipases were obtained from SIGMA-ALDRICH (St. Louis, MO, U.S.A.). After adding the lipases, the biosensors were monitored after one hour at 30.0° C. to evaluate any color change. Additionally, to assess the effect of time on color change, Amano Lipase PS from Burkholderia cepacia was selected for further testing. The membranes were monitored at 15-minute intervals (30.0° C.) for one hour at various concentrations (0, 0.25, 0.5, 0.75, 1, 3, and 10 mg/mL).

### Bench Besting the Biosensors

To ensure the biosensors' responsiveness to lipase and minimize the occurrence of false positives, a bench test was designed as a final quality control measure before proceeding to bacterial testing. In this test, biosensors (1.0×1.0 cm²) were placed in either pure water or AWF solution with or without the addition of lipase (500 μL, Lipase PS from Burkholderia cepacia, 10 mg/mL). The samples were incubated at 40.0° C. for 30.0 minutes. Following incubation, the color of the membranes was observed, and digital photographs were taken under different lighting conditions (daylight, cool light, and biosafety cabinet light) at 45° and straight angles. Additionally, the membranes' color was quantitatively evaluated using a spectrophotometer (GretagMacbeth ColorEye 2180UV), and CIELAB (C2°) data, including L*, a*, and b* values, were recorded. The corresponding color was also identified using the HEX #color code as a control reference.

### Bacteria Response

The color change of the biosensors in the presence of bacteria was evaluated using three different bacterial strains: MRSA, E. coli, and P. aeruginosa. Bacterial plates were prepared to achieve a theoretical concentration of 10⁵ CFU/cm² based on previously reported procedures. Briefly, 57 μL of a 10⁸ CFU/mL bacterial solution was spread on each plate and incubated at 37° C. for 1 hour. Following incubation, biopsy punches were taken from all plates to determine the initial bacterial concentration. For each bacterial strain, three biological replicates were tested, and for each biological replicate, three technical replicates were used for bacterial counting. Once the membranes exhibited a color change to green, they were placed on a glass slide, and the color data (L*, a*, b*) were measured using a spectrophotometer. These values were then converted to a standard color based on the HEX #code. Additionally, the final bacterial concentration was determined using a biopsy punch.

Compatibility of the Biosensor with Commercial Foams

Given that the biosensors are intended for commercial application and might not be directly applied to wounds, the biosensors of the present invention were evaluated for their compatibility with various foam dressings covered with a commercially available transparent adhesive film, Tegaderm®. Nine different types of foams were tested: AQUACEL® Foam (Convatec Forever Caring), Curafoam Dressing (Dynarex®), HEALQU® Non-Adhesive Waterproof Foam, Hydrofera Blue Classic®, Mckesson® Hydrocellular Foam Dressing Non-Adhesive, Medline® Optifoam-Basic, Medline® Optifoam Non-Adhesive, Medline® Quick, and PolyMem® Non-Adhesive Pad.

The compatibility of the biosensors with these foams was assessed based on color change in the presence of commercial lipase and during bacterial tests. For the lipase test, the amount of lipase solution used was determined by the swelling capacity of each foam. The swelling capacity was measured by incrementally adding the solution and weighing the foams until a plateau was reached.

Two approaches were employed for the bacterial tests. In the first approach, the foam was placed on top of the agar without any additional solution. In the second approach, a wound-like environment was simulated by curving the agar, placing the foam inside the agar, and adding Simulated Wound Fluid (SWF), which comprised 50% fetal bovine serum (FBS) and 50% peptone water. The peptone water (0.1% peptone, 0.9% NaCl) was autoclaved for sterilization before being mixed with FBS. Furthermore, to confirm that the observed color change was explicitly due to the presence of bacteria in direct dry contact with the biosensors, the foams and biosensors were examined using SEM. This analysis was conducted to verify the presence of bacteria on the surface of the biosensors.

Compatibility of the Biosensor Use with a Wound-Cleaning Solution

The performance of the biosensors provided by the present invention was further evaluated for compatibility with Hypochlorous Acid (HPA), a common wound-cleaning solution. Agar plates were prepared as previously described. After placing the biosensors on the agar, the plates were incubated overnight to allow for a high concentration of bacteria to develop (˜10⁴ CFU/cm²). The following day, each plate was divided into four sections. The first quadrant served as a bacteria control to determine bacterial concentration, while the second quadrant was splashed with 1 mL of HPA. The third quadrant was cleaned using gauze soaked in HPA, and the fourth quadrant served as a biosensor control. Following these treatments, a new biosensor was placed on each quadrant, and the color change performance was monitored over time to ensure that the presence of HPA did not interfere with the biosensors. If a color change was observed, a biopsy punch was used to determine the bacterial concentration at that specific time point.

Performance of Biosensors after Sterilization

In addition to evaluating the biocompatibility of the biosensors provided by the present invention for commercial use, it is crucial to assess their performance following sterilization. E-beam sterilization was selected as the appropriate method, with three different intensity levels (low, medium, and high doses) applied for the sterilization process. The sterilized samples were subjected to bench testing and compared to non-sterilized samples. Furthermore, the bacterial performance of the sterilized samples was evaluated using the previously described bacterial testing procedure. This comparison with non-sterilized samples ensured that the E-beam radiation did not compromise the functionality of the colorimetric probe.

### Leaching Test

Given the potential for the dye to leach into the environment after cleavage, the leachability of the biosensors was assessed using different foams in two separate conditions. In the first test, 500 μL of lipase solution was added to the foams, while in the second test, the foams were immersed in 5 mL of the solution. Both setups were maintained overnight. Subsequently, the foams were dried in an oven at 40° C., sectioned, and the depth of dye penetration into the foam was measured.

### Cell Viability

The biocompatibility of the biosensors provided by the present invention was evaluated using the MTT assay (a colorimetric assay used to measure cell metabolic activity), following ISO 10993-5 guidelines. Sterilized samples, each with an area of 2 cm×2 cm, were immersed in 333 μL of complete fibroblast media (HLL Supplement: HSA 500 μg/mL, linoleic acid 0.6 mM, lecithin 0.6 μg/mL, L-Glutamine 7.5 mM, rh FGF basic 5 ng/ml, rh EGF/TGF-1 Supplements 5 ng/ml and 30 μg/mL, rh Insulin 5 μg/mL, Hydrocortisone 1 μg/mL, Ascorbic acid 50 μg/mL, antibiotics (Penicillin-Streptomycin-Amphotericin B Suspension), and 5% FBS). Five replicates were prepared for each sample. The samples were incubated at 37° C., 5% CO2, and 95% humidity for 72 hours to allow for extraction.

For cell preparation, 100 μL of complete growth media was added to each well of a 96-well plate, followed by the addition of 100 μL of a 1.0×10⁵ cells/mL suspension, resulting in a final concentration of 1.0×10⁴ cells/well. The cells were incubated under the same conditions for 24 hours. After incubation, the media was replaced with the extracted solutions (in four dilutions: 1.0, 0.1, 0.01, and 0.001×), with DMSO serving as a negative control and media as a positive control.

After a further 24 hours of incubation, the extracts were removed, and an MTT assay was performed. Fresh media (100 μL) was added to each well, followed by the addition of 10 μL of 12 mM MTT stock solution (5 mg in 1 mL PBS). The cells were incubated for 3 hours, after which the media was removed, leaving 25 μL in each well. Subsequently, 50 μL of DMSO was added to dissolve the formazan crystals. The mixture was pipetted up and down and incubated for 10 minutes. Finally, the absorbance of each well was read at 540 nm, and cell viability was determined by comparing the optical density (OD) of each sample to that of the control (untreated cells).

### Nanoparticle Fabrication

After successfully fabricating and examining biosensors provided by the present invention, a method was developed to fabricate and incorporate into the biosensors ROS responsive nanoparticles loaded with an antimicrobial. For a preferred embodiment of the present invention ciprofloxacin was selected. A responsive polymer was synthesized, and then NPs were fabricated using an emulsion technique.

Synthesizing Methoxy Poly(Ethylene Glycol) Methacrylate (mPEG45 Methacrylate)

mPEG (1.0 g), triethylamine (0.6 mL), and methacryloyl chloride (1.0 mL) were added into dichloromethane 20 mL, and the reaction continued at room temperature for 48 hr. Thereafter, the rotary evaporator was used to concentrate the solution, followed by filtering and precipitating the products with cold diethyl.

Synthesizing Methoxy Poly(Ethylene Glycol) Thioacetate (mPEG45 Thioacetate)

mPEG methacrylate (0.2 g) was dissolved in 5 mL THF and was stirred for an hour. Then, AIBN (0.0065 g) and thioacetic acid (110 μL) were added to the solution, and the reaction chamber was filled with nitrogen gas for 30 seconds. After that, the reaction chamber was degassed by freezing the mixture under liquid nitrogen, evacuating it under a high vacuum, and filling it with nitrogen. The freezing-pumping-thawing cycle was redone until no bubbles formed at the thawing stage. The reaction chamber was heated to reach room temperature, and then the solution was mixed for 24 h at 60° C. After 24 hours, the chamber was placed in the rotary evaporator. The final step was filtering and precipitating by cold diethyl ether to obtain powders. The obtained powder was used for chemical characterization and future steps [1].

Synthesizing of Methoxy Poly(Ethylene Glycol)-b-Poly-(Propylene Sulfide) (mPEG45-b-PPS60)

mPEG thioacetate (0.37 g) was dissolved in 10.0 mL THF under nitrogen gas in the Schlenk tube. Then sodium methoxide (0.012 gr in 445 μL methanol) was added to the solution by a syringe and stirred for 30 min at room temperature. Then propylene sulfide (0.87 mL) was added to the mixture and stirred overnight at 60° C. After 24 hours, the solvent was removed using the rotary evaporator, and the final product was obtained by precipitating with cold diethyl ether and filtering. The dried powder was placed in a dialysis tube (12-14 kDa cut off) filled with ultrapure water (30 mL) and immersed in 500 mL ultrapure water for 48 hr at continuous stirring. The medium water was changed frequently. After 48 hr, the solution was transferred to two 50 mL tubes and placed in the freezer for freeze-drying.

For each step of synthesizing, NMR and FTIR were used to characterize the final product chemically, and the yield of each step was calculated using the following formula.

$$y ield of synthesize = Final Weight of obtained Product ( g ) / Theorical Weight of final Product ( g )$$

### Fabrication of NPs and Drug Loading

The synthesized PEG-co-PPS (80 mg) was used to fabricate the ROS-responsive NPs with different ciprofloxacin (CIP) contents (6.25, 39, and 244 mg in 1 mL PBS 7.4). The drug solutions were added dropwise to the polymer solution (5 mL Chloroform), and the final solution was sonicated for 3 min in an ice bath, 20 W. Then, the obtained emulsion was added dropwise to the 20 mL PBS (containing 0.5% PVA) while mechanically stirring (1000 rpm). The emulsion was sonicated for 1 min in an ice bath, 40 W. The rotary evaporator was used to remove the oil phase of the obtained emulsion. The final product was centrifuged to obtain the nanoparticles. Transmission Electron Microscopy (TEM) with negative staining was employed to evaluate the morphology of the NPs, while dynamic light scattering (DLS) was used to determine their hydrodynamic diameter. Also, the NPs were exposed to H₂O₂ (0.5 mM) to evaluate the changes in morphology with TEM. The stability of the NPs in PBS buffer (pH=7.4, T=4° C.) was also assessed after three months of storage with DLS.

### Loading NPs in the Nanofibers

The NFs (10 cm×10 cm) were immersed in the 2 mL NPs solution (200 mg/mL) overnight and then washed three times with PBS to eliminate the non-loaded NPs and were dried in a vacuum oven (37° C.) overnight. The dried NP-loaded NFs were used for morphology characterization by SEM.

Drug Loading Efficiency and Drug Release Behavior from NPs

0.6 mg/mL of NPs was treated with 100 μm H₂O₂ (30%) overnight and used for UV absorbance at 325 nm to find the loading efficiency (LE) and loading capacity (LC) of ROS NPs based on the following equations.

$$EE \% = ( \frac{Initial added drug - Free drug}{Initial added drug} ) \times 100 \qquad Equation 1$$

$$LC \% = ( \frac{Totall entraped Drug ( mg )}{Totall NPs weigth ( mg )} ) \times 100$$

10 mg of drug-loaded NPs and 1 cm×1 cm NP-loaded NFs in 2 mL PBS were used for the release profile in the absence and presence of H₂O₂ (1 mM) as a reactive oxygen agent. The suspensions were placed in a dialysis tube and immersed in 23 mL PBS. At each interval, 1 mL of solution was collected and replaced with fresh PBS. The collected solution was used for absorbance reading at 325 nm. Different release profiles, zero-order, first-order, Korsmeyer-peppas, and Higuchi, were used to fit the release data using the formula in Table 1.

TABLE 1
The release profiles

| Model | Formula |
|---|---|
| Zero-order | Mₜ = M₀ + Kt |
| First-order | In Mₜ = ln M₀ + Kt |
| Korsmeyer-Peppas | Mₜ/M_{∞} = Ktⁿ |
| Higuchi | Mₜ/M_{∞} = K√t |

### Antibacterial Tests

The antibacterial properties of the fabricated NPs and NFs and NP-loaded NFs were assessed using different techniques.

The Minimum Inhibitory Concentrations (MICs) and Minimum Bactericidal Concentrations (MBCs)

The MICs and MBCs of the NPs were determined for three different bacterial strains: E. coli, P. aeruginosa, and MRSA. MICs are defined as the lowest concentration of an antimicrobial agent that inhibits visible microbial growth after overnight incubation, while MBCs refer to the lowest concentration that prevents growth after subculturing onto antibiotic-free media. These measurements were conducted both in the presence and absence of H₂O₂ (0.5 mM), with ciprofloxacin (CIP) at varying concentrations used as a control. The MIC of the antimicrobials was determined using the microdilution method in culture broth, following the guidelines set by the Clinical and Laboratory Standards Institute (CLSI) of the United States. A range of NP and Cip concentrations (128-0.06 μg/mL) were tested. The antimicrobial components were added to sterilized Mueller-Hinton broth to prepare different concentrations.

To prepare the bacterial suspension, a single colony of each bacterial strain was inoculated into 3 mL of Mueller-Hinton broth and incubated at 37° C. for 18 hours. The turbidity of the suspension was then measured using the McFarland standard and adjusted to 0.5, corresponding to approximately 10⁸ CFU/mL. Following the preparation of bacterial and component suspensions, 50 μL of each concentration was added to the wells of a 96-well plate, followed by the addition of 50 μL of bacterial suspension (reaching 5×10⁵ CFU/mL). A sterility control, consisting of 100 μL of broth without bacteria, was included as a positive control, while a growth control, containing 50 μL of bacterial suspension and 50 μL of broth without any antimicrobial agents, served as a negative control. The plates were then incubated overnight at 37° C. The MIC was determined by identifying which of the wells had the lowest concentration of the antimicrobial component that exhibited a clear solution, indicating inhibition of bacterial growth, while the sterility control remained clear, and the growth control showed turbidity.

To determine the MBC, the wells that exhibited no visible bacterial growth from the MIC assay were selected for further analysis. 100 μL from these wells was plated onto Mueller-Hinton agar and incubated at 37° C. overnight. The MBC was identified as the lowest concentration of the antimicrobial agent, which resulted in no bacterial growth on the agar plates.

### Zone of Inhibition (ZOI)

Biosensors were punched into discs using a 6.0 mm biopsy punch to evaluate the ZOI. Three types of biosensors were tested: one group without citric acid in the shell, a second group with citric acid in the shell, and a third group loaded with NPs. Bacterial plates were prepared by adjusting the inoculum concentration to 10⁸ CFU/mL (equivalent to 0.5 McFarland standard). A cotton swab was dipped into the bacterial suspension, and after removing excess broth by pressing the swab against the tube's wall, the swab was used to streak the plate three times, rotating it 60 degrees after each streak. After a five-minute wait, the prepared discs were placed on the agar with a minimum distance of 24 mm between them. For each bacterial strain, three sets of plates were prepared: one set without hydrogen peroxide (H₂O₂), another set with H₂O₂ (1 mM) added to the broth before plating, and a third set where the biosensors were dipped in 1 mL of H₂O₂ (1 mM) for 2 seconds prior to placement on the agar. The plates were then incubated at 37° C. overnight, and the clear zones surrounding each disc were measured the following day.

### Direct Antibacterial Test

In this antibacterial test, 200 μL of bacterial suspension (10⁵ CFU/mL) was added to different biosensors (6.0 mm in diameter), with or without the addition of H₂O₂ (1 mM), and incubated at 37° C. for 4 hours. After incubation, the membranes were rinsed and vortexed to detach any adhered bacteria. The resulting suspension was then subjected to serial dilution plating to determine the bacterial concentration. For each sample, the bacterial concentration was compared to the control (with or without H₂O₂), and the log reduction in the bacterial count was calculated.

### Time Dependent Antibacterial Test

Three bacterial strains were prepared with an initial concentration of 10⁶ CFU/mL. Samples, including CIP, NPs, and NP-loaded NFs, were added to 3 mL of the bacterial suspension with and without H₂O₂, at a concentration equivalent to ¼ of the MIC for both CIP and NPs. Bacterial concentrations were determined at 0, 5, and 24 hours using sampling and serial dilution plate counting. These concentrations were then compared to the controls, and log reduction was calculated to evaluate the bactericidal efficacy over time.

### Cytocompability

The biocompatibility of the final biosensors containing ROS-responsive NPs was assessed using various tests involving the MTT assay kit and human fibroblast cells.

### Cell Culture

Human primary dermal fibroblasts (ATCC® PCS-201-012) were cultured in Fibroblast Basal Media supplemented with Fibroblast Growth Kit and 100 g/mL antibiotics cocktail at 37° C. in a 5% CO₂ atmosphere. Cells were harvested with the Trypsin enzyme before the cellular test. Briefly, cells were washed with cold PBS three times, and then Trypsin was added. Cells were incubated at 37° C. for 5 minutes and then transferred to a tube containing complete media. The cell suspensions were centrifuged for 10 minutes at 1,000 g, and the cell pellet was resuspended in a complete media. The resuspended cells were used for cell counting. 20 μL of cells were mixed with 20 μL of Trypan blue solution, and 10 μL of cell suspension was used for cell counting. The suspension was added to the hemocytometer, and the number of live cells was found. The procedure was done three times for each cellular test.

IC50 for Cip, NPs with and without H₂O₂

To assess the cytocompatibility of the ROS-responsive NPs, various concentrations of Cip, NPs, and NPs exposed to 1 mM H₂O₂ overnight were tested. The concentrations tested for NPs were 0, 0.5, 1.0, 1.5, 2.0, 5.0, 10.0, and 100 mg/mL. For CIP, concentrations were selected based on the LC % of the NPs and included 0, 0.18, 0.36, 0.54, 0.72, 1.8, 3.6, and 36 mg/mL. The IC50 value, which represents the concentration at which 50% of the cells were dead, was determined for all samples.

A suspension of 2×10⁵ cells was added to each well in complete media and incubated for 24 hours to allow for complete adhesion. The following day, the media was replaced with fresh media containing the test samples (Cip, NPs, and NPs exposed to H₂O₂), and the cells were incubated for an additional 24 hours. Subsequently, the MTT assay kit was used to quantify cell viability, following the manufacturer's instructions.

### Cytocompability of NP-Loaded NFs

The biocompatibility of biosensors loaded with ROS-responsive NPs was evaluated both in the absence and presence of H₂O₂ using the elution technique. A 1 cm×1 cm membrane was immersed in 5 mL of complete media for three days, with or without H₂O₂, and the resulting extraction was applied to the cells as described previously. Cell viability was assessed on days 1, 3, and 7, and the results were compared to a control group without any treatment.

### A Mixture of Bacteria and Cells

A total of 1×10⁵ cells were added to each well and incubated overnight. Diluted bacterial suspensions in complete culture media (10³ CFU/mL) were then prepared and added to each well at a ratio of 1:10, followed by incubation for 4 hours. After this period, 100 μL extracts from NP-loaded NFs, both with and without H₂O₂ treatment, were added to each well and incubated overnight. A positive control was included by adding a cocktail of antibiotics to one well, while a negative control consisted of a well without any treatment. Additionally, a growth standard consisting of non-infected cells was used. After 24 hours, the cell monolayer was lysed with Triton X-100, and the suspension was diluted for serial dilution plating to count bacterial colonies. Furthermore, the number of live cells was assessed using trypan blue after thoroughly washing the cell monolayer with cold PBS and trypsinizing.

### Electrospinning and Morphological Characterization of the Biosensors

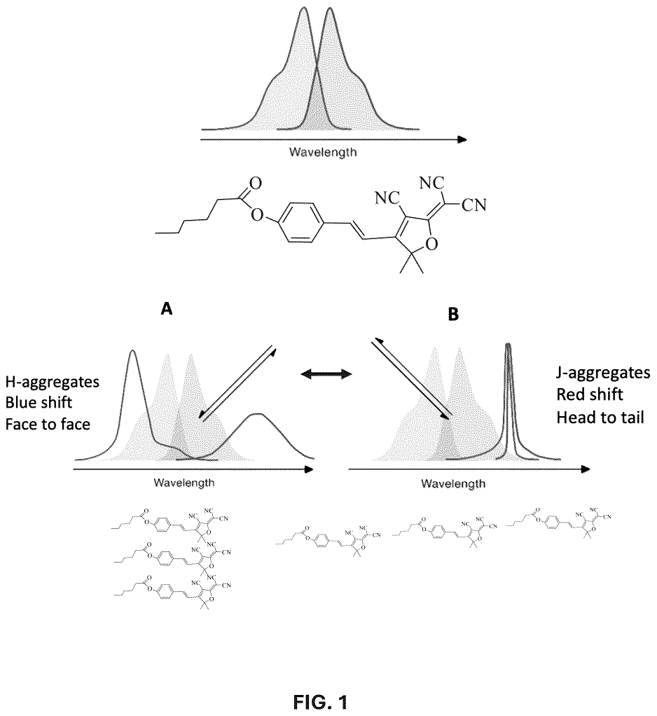

Referring to FIG. 1, it shows how different aggregate configurations of the dye were found to cause different color changes in the biosensor of the present invention. Electrospun membranes were cut and coated to evaluate the nanofiber morphology, fiber diameter, and biosensor thickness. The thickness of the biosensor was determined to be critical to its performance, as optimal thickness is necessary to prevent false positives. False positives can arise due to various environmental factors, such as increased pH or the presence of mixed salts at specific concentrations. These factors affect the aggregation behavior of the dye and alter the way electrons circulate within these aggregates. When dye monomers aggregated in a face-to-face configuration they form H-aggregates, leading to a blue shift in the spectrum. Conversely, when the interaction is head-to-tail, J-aggregates are formed, resulting in a redshift. These different configurations modify intramolecular interactions, which were determined to be highly dependent on the packing arrangement of the dye molecules, ultimately causing a color change in the system.

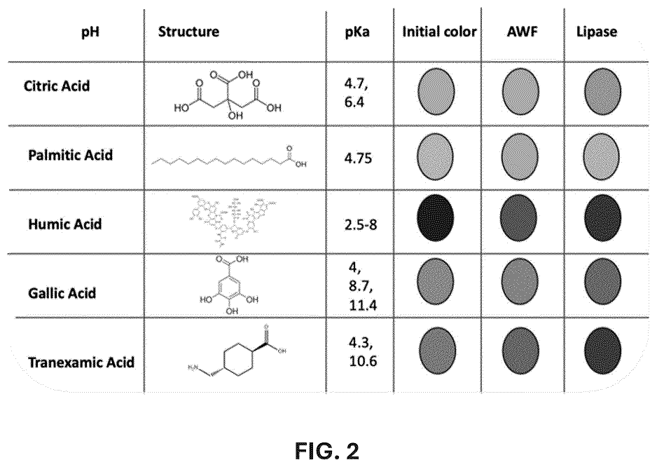

FIG. 2 shows the effect of the addition of different acids to the biosensors and performance impact in AWF solution with and without lipase. To neutralize environmental factors and prevent false positives, various additives were introduced as a remedy. The chosen additives for the biosensor in this project were acids known for their wound healing properties: citric acid, palmitic acid, humic acid, gallic acid, and tranexamic acid. These acids may react with excess salts in the medium, neutralizing them and preventing changes in the dye's aggregate configuration. After the addition of each acid, a quick test was performed in AWF at pH 8.0 to ensure false positives did not occur. The initial color of the membranes varied based on the added acid: citric and palmitic acids produced a yellow color, humic acid black, gallic acid orange, and tranexamic acid red. Following the addition of AWF without lipase at pH 8.0, the membrane containing citric acid did not show any color change. In contrast, palmitic acid turned green, humic acid brown, gallic acid dark orange, and tranexamic acid brown. Among all the samples, unexpectedly only the membrane with citric acid exhibited a distinct color change in the presence of lipase, indicating that the other acids interfered with the membrane's lipase responsiveness. Consequently, citric acid, which showed the most promising results in preventing false positives, is used in a preferred embodiment of the present invention for the fabrication of market-ready biosensors.

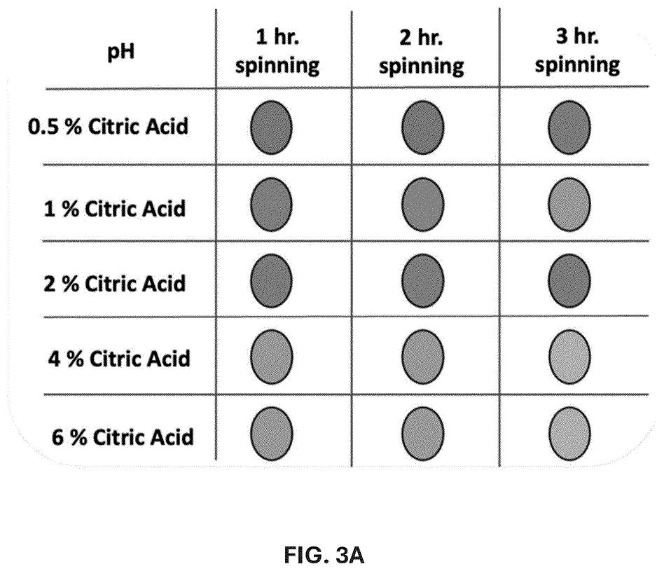

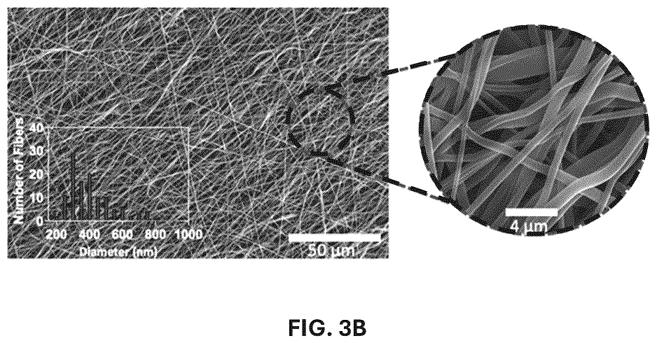

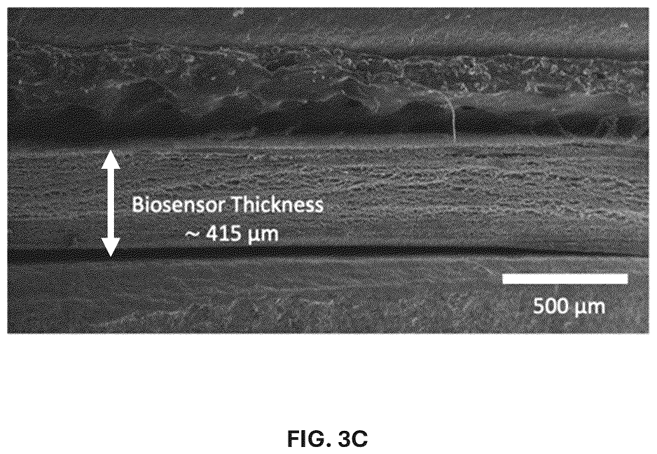

FIG. 3A shows the results of biosensor testing under different spinning times and citric acid concentrations in the presence of AWF (pH 8.0). In regard to preventing false positive color change by the addition of acid to the biosensor, the nanofiber diameter and the thickness of the biosensors was determined to be of paramount importance. The nanofiber diameter and thickness of the biosensor affects the amount of citric acid that can be loaded into a membrane. As diameter and thickness increases, more citric acid can be incorporated into the nanofibers, which can enhance the biosensor's performance and accuracy but also pose challenges during extended electrospinning, potentially disrupting continuous spinning. Moreover, citric acid, being a potent antibacterial agent, can also hinder the biosensor's sensitivity by killing bacteria upon contact. To determine the optimal parameters for biosensor fabrication, different citric acid concentrations (0.5-6%) and electrospinning times (1-3 hours), resulting in thicknesses ranging from 50 to 500 μm, were tested in the presence of AWF at pH 8.0. The ideal parameters were determined to be 4% citric acid (as higher concentrations did not improve performance), a spinning time of 3 hours (since shorter times led to false positives), and a thickness of approximately 350-450 μm. The results of biosensor testing under different spinning times and citric acid concentrations in the presence of AWF (pH 8.0).

Referring to FIG. 3B, it shows the morphology of a preferred embodiment of a biosensor provided by the present invention. A SEM image and fiber diameter distribution in a biosensor of the present invention is presented. The biosensors fabricated with preferred parameters were further analyzed for fiber diameter and distribution. The fiber diameters ranged from 200 nm to 800 nm, an average diameter of 412±140 nm with highest frequency at 240 nm.

Referring to FIG. 3C, it shows a SEM image from the cross-section of the biosensor showing the thickness of the biosensor. The best-performing sample was selected for further testing in various solutions and salt concentrations to ensure that no false positives occurred. The membrane thickness in a preferred embodiment on average was approximately 415 μm.

### Response to Different Solutions

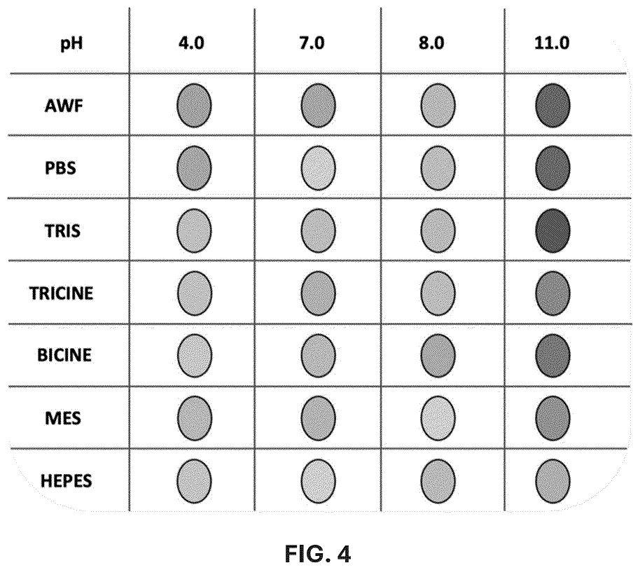

Referring to FIG. 4, it shows color-changing response of the biosensor in the presence of different buffers at different levels of pH. To ensure the accuracy and precision of the fabricated biosensor under realistic wound conditions, which are rich in proteins and salts, various buffers, salts, and protein solutions at different concentrations and pHs were tested. The results indicated that no false positives were observed in the presence of various buffers, including AWF, PBS, Tris, Tricine, Bicine, MES, and HEPES, at pH levels of 4.0, 7.0, and 8.0. However, at pH 11.0, all buffers caused a color change from yellow to green due to ester bond hydrolysis, which enhances internal charge transfer and facilitates the color shift, as depicted.

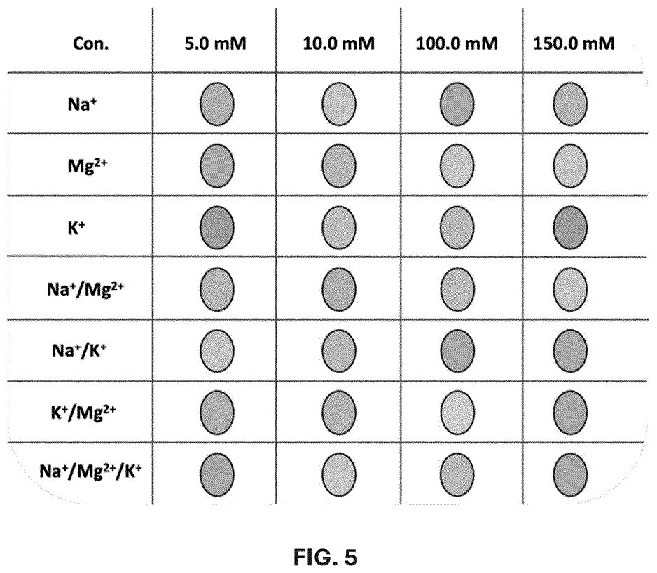

Referring to FIG. 5, it shows color-changing response of the biosensor in the presence of different ions at different concentrations. The presence of ions (Na⁺, Mg²⁺, and K⁺), whether alone or in combination, did not induce any color change towards green across concentrations ranging from 5.0 mM to 150.0 mM. These results confirm that the fabricated biosensor does not produce false positives in response to different ions and buffers.

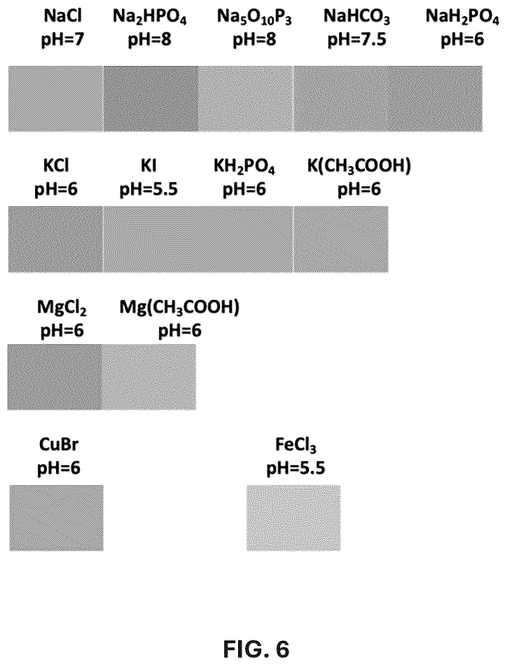

Referring to FIG. 6, it shows color-changing response of the biosensor in the presence of different salts. To further evaluate the sensor's selectivity, various salts of these ions were also tested, including NaCl, Na₂HPO₄, Na₅P₃O₁₀, NaHCO₃, NaH₂PO₄, KCl, KI, KH₂PO₄, K(CH₃COOH), MgCl₂, Mg(CH₃COOH), CuBr, and FeCl₃. No green color change was observed with any of these salts, demonstrating the biosensor's selectivity for lipase.

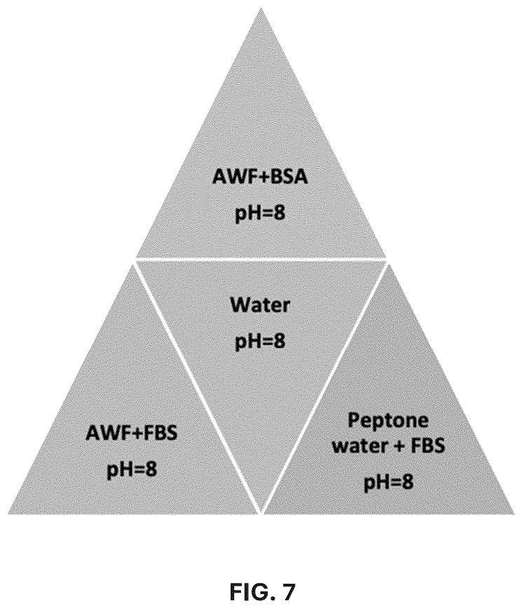

Referring to FIG. 7, it shows color-changing response of the biosensor in the presence of different proteins. Testing in the presence of proteins such as FBS and BSA in different buffers also yielded no color change, showing that the biosensor does not react with proteins. These findings highlight the biosensor's potential for specific bacterial detection, with no false positives from other environmental factors, underscoring its reliability.

### Response to Different Lipases and Sensitivity of the Biosensor

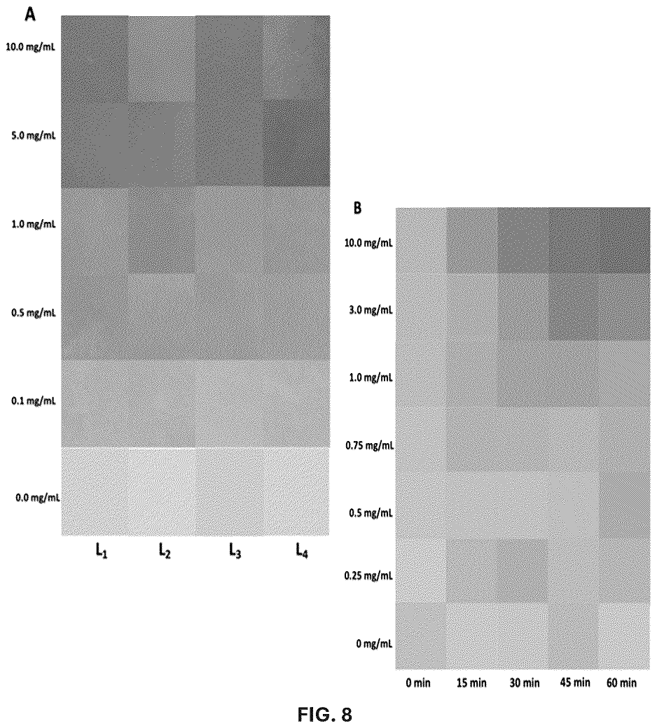

Referring now to FIG. 8, it shows biosensors color changing A) after one hour in the presence of different lipases from bacteria and fungi sources; and B) during one hour at different concentrations of L₁. To assess the response of the biosensor, various lipases from different sources were selected. Additionally, to determine the biosensor's sensitivity, different concentrations of each lipase were tested. As shown as 8A, after 1 hour of exposure to different lipase concentrations—Amano Lipase PS from Burkholderia cepacia (L₁), Amano Lipase from Pseudomonas fluorescens (L₂), Lipase from Pseudomonas cepacia (L₃), and Lipase from Candida rugosa (L₄)—all biosensors exhibited a noticeable color change. At the lowest concentration of 0.1 mg/mL, the color change was subtle, but at 0.5 mg/mL, all lipases induced an orange color visible to the naked eye. Although L₄, derived from fungi, caused a color change, it was less pronounced compared to the bacterial lipases. These results indicate that the biosensor can detect various lipases, even at low concentrations like 0.1 mg/mL, though higher concentrations produce more distinct color changes.

To further evaluate the biosensor's performance, Li was chosen to assess the effect of time on color change at a fixed concentration, shown as 8B. The color change occurred almost instantly at the highest concentration (10 mg/mL). As the concentration decreased, time became a more critical factor in the color change process, as the dye concentration exceeded that of the available lipases at each time point. For all concentrations tested, a color change was observed within 1 hour, demonstrating the biosensor's sensitivity.

### Bench Testing the Biosensors

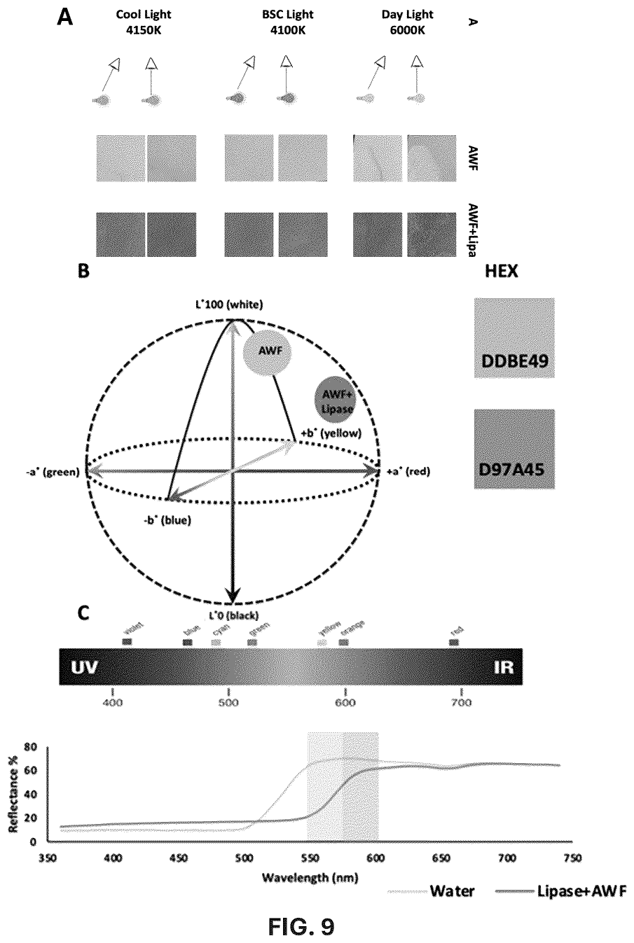

Referring to FIG. 9, it shows A) bench test study of biosensors color in the absence (AWF) and the presence (AWF+lipase) of lipase under different lighting settings and at two different angles; B) the color space and color of biosensors in AWF and AWF+lipase; and C) the reflection spectra of AWF and AWF+lipase samples indicate the differences between these two samples. As a quality control step, a bench test was designed to ensure the biosensors produced do not yield false positives in the absence of lipase and reliably exhibit a color change in its presence. Since the biosensor is intended for various settings, including hospitals, home care, battlefields, and household use, it was important to assess the visibility of the color change under different lighting conditions. As shown as 9A, varying light temperatures slightly influenced color perception, but the differences were not significant. Similarly, the angle of observation did not noticeably affect the visibility of the color change. These findings were consistent for both biosensors in AWF (no lipase) and AWF with lipase. The biosensors in AWF and AWF with lipase were also analyzed using spectrophotometry to determine their L*, a*, and b* values and to generate reflectance graphs. As shown as 9B, the AWF samples exhibited a yellow color with a positive b* value (L*=78.2, a*=1.8, b*=66.72), while the AWF with lipase samples shifted towards the red spectrum with a positive a* value (L*=61.3, a*=34.9, b*=44.8). The L*, a*, and b* data were used to generate HEX color codes for reference in future bench tests as part of quality control during mass production. As shown as 9C, the clear distinction between the two samples is illustrated in terms of reflectance and wavelength. The AWF sample had a reflection peak at 550 nm, corresponding to the yellow range, while the AWF with lipase peaked at around 600 nm, indicating an orange hue. This orange color results from a combination of cleaved dye (red) and non-cleaved dye (yellow), confirming the biosensor's functionality.

### Bacteria Response

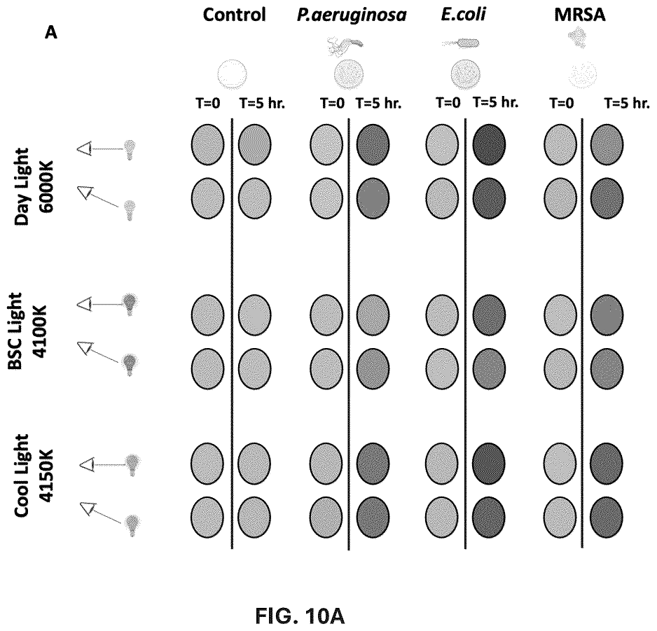

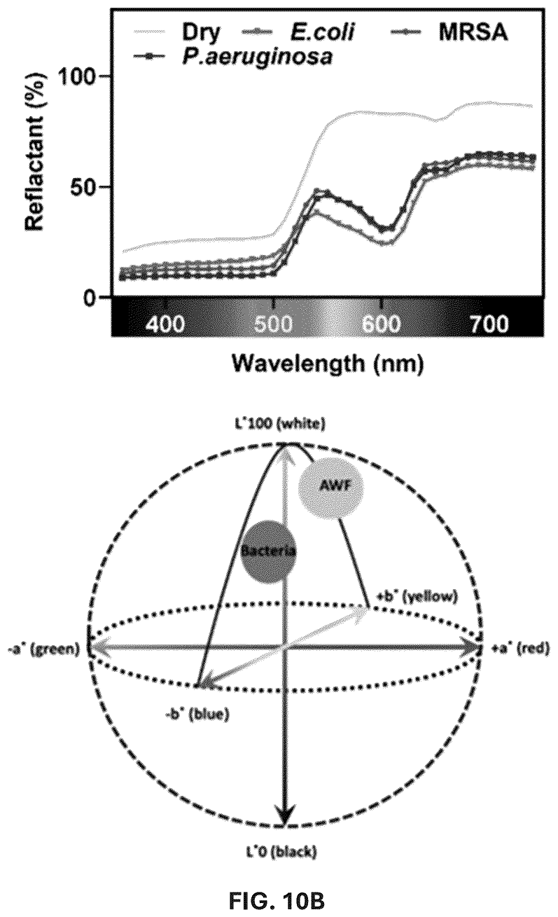

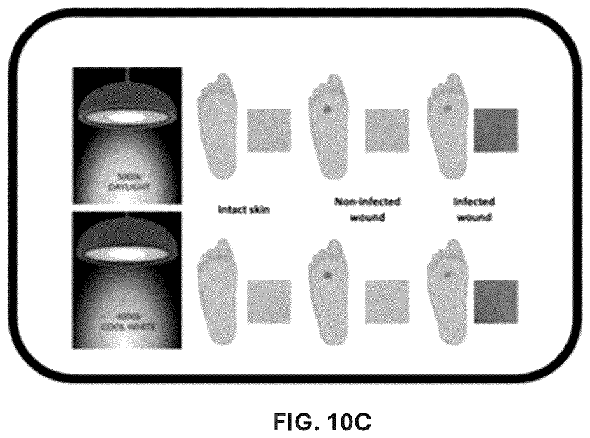

Referring to FIG. 10A, it shows the performance of the biosensors of the present invention in the presence of different bacteria, the color changing of the biosensor in the presence of P. aeruginosa, E. coli, and MRSA under different light sources compared to control with no bacteria, and the flashcard for differentiating infected wounds from non-infected wounds based on the color change. The performance of the biosensors in detecting bacteria was assessed using the agar method. To ensure the observed color changes fell within an acceptable range, the same lighting conditions as those used in the bench test were applied. The color was examined both directly at eye level and at a 45° angle. The biosensors exhibited a noticeable color change for all three tested bacteria (P. aeruginosa, E. coli, and MRSA). However, the color change appeared slightly different under varying light sources. All changes were clearly distinguishable from the control, which showed no color change. The color shifts occurred within 5 hours of sample placement, a timeframe suitable for clinical applications. The initial bacterial concentration was 3×10⁵ CFU/cm², but after 5 hours, significant growth was inhibited due to the presence of citric acid, which has antibacterial properties. Following the 5-hour incubation, the biosensors were used to isolate and culture the bacteria to further assess bacterial concentrations in the presence of the biosensor. The bacterial concentration around the biosensors remained close to the initial level, between 5-6×10⁵ CFU/cm².

Referring to FIG. 10B, it shows the color wavelength spectra of biosensors after exposure to the bacteria and control and the color space of the control and bacteria exposed sample. The color-changed biosensors were also analyzed using spectrophotometry to determine their color spectra. As shown, a shift toward the green spectrum was observed at a wavelength of 525 nm, compared to the control sample, which showed a yellow color at 550 nm. In terms of color space, the green-shifted biosensors had L* values between 66-72, b* values between 42-48, and negative a* values ranging from −3 to −6, indicating a shift toward green.

Referring to FIG. 10C, it shows a flash card developed to help end users differentiate between infected and non-infected wounds by showing the color changes associated with bacterial presence under different light sources, based on results as presented in FIG. 10B.

Compatibility of the Biosensor with Commercial Foams

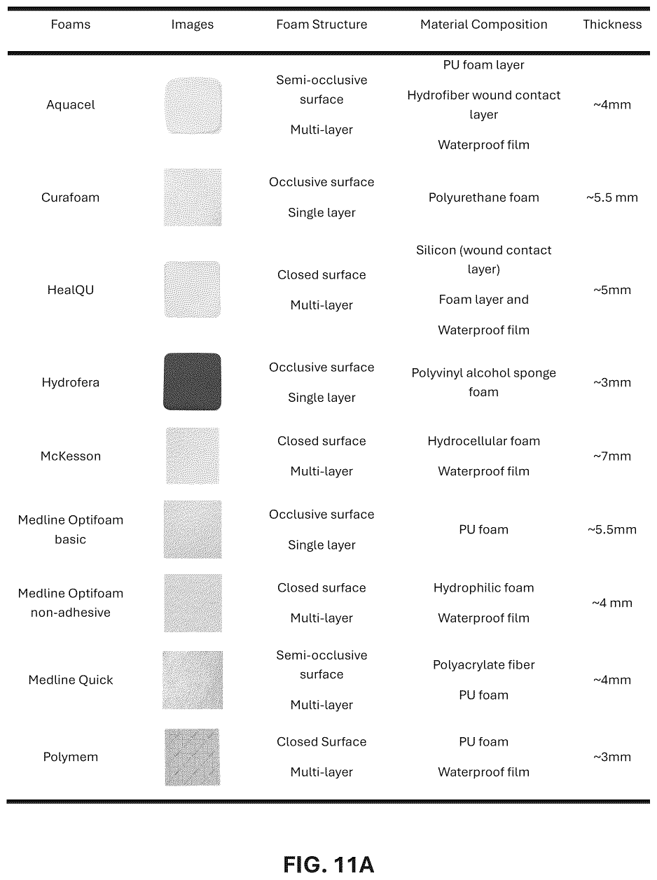

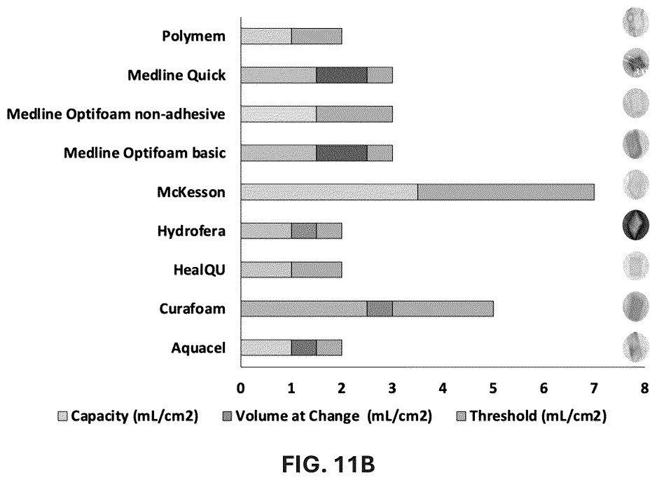

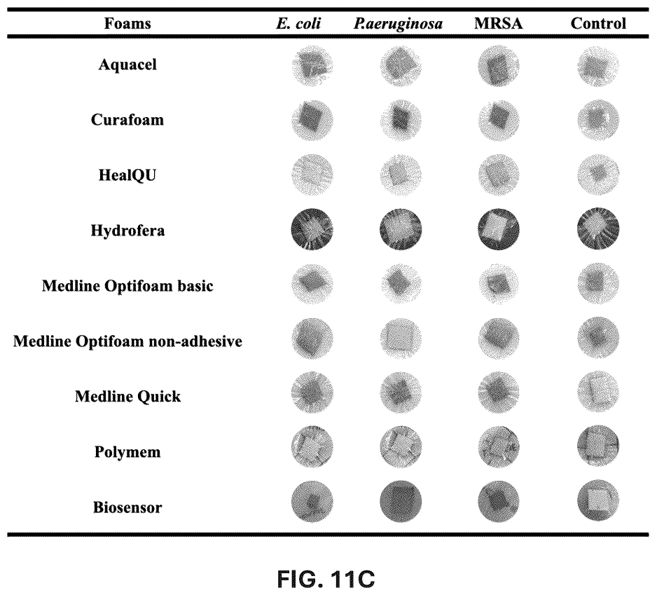

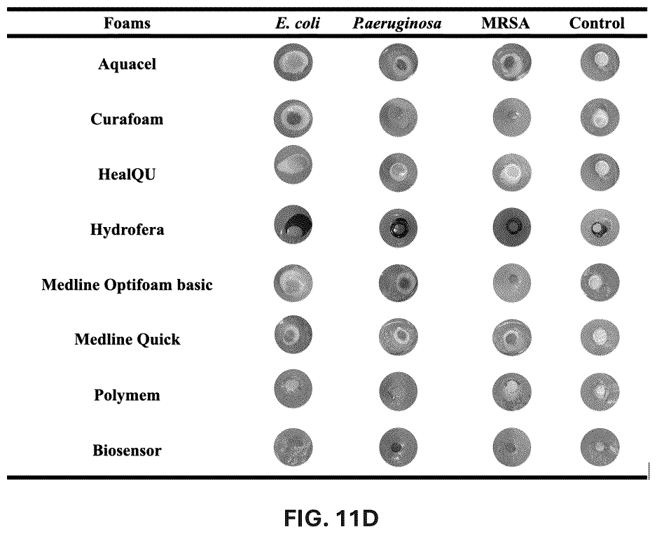

Referring to FIG. 11A, the properties are shown for commercially available foam tested for compatibility with a preferred embodiment of the biosensor of the present invention. Based on the clinical use and circumstances, the fabricated biosensors might not be used directly on the wound. Therefore, we tested the compatibility of the fabricated biosensors with different commercially available foam products to indicate that a color change would happen even when not placing the biosensor directly on a wound. The name and properties of these foam products are provided as indicated.

In a preferred embodiment, the color change mechanism of the biosensors relies on the presence of lipase, so the penetration of water from a foam to the biosensor is critical. However, this presents a challenge regarding an excess of wound fluid. The wound fluid, which is rich in salts, could potentially trigger a false positive. To address this, a bench test was designed to evaluate the water uptake capacity of various foams, determine the minimum volume required to induce a color change in the presence of lipase, and identify the amount of excess fluid that causes a false positive for each foam.

Referring to FIG. 11B, the results of this bench test are shown, where the displayed colors represent the actual biosensor responses. As indicated in the figure, several biosensors did not show any orange color change in the presence of lipase, including Polymem, Medline Optifoam Non-Adhesive, Mckesson, and HealQU. The common feature of these foams is the presence of a protective waterproof layer, which prevents lipase from interacting with the biosensor when placed atop the waterproof layer. Thus, no color change occurs. For the remaining foams, a positive color change was observed before reaching the threshold for a false positive to occur. Based on these results, the foams tested were classified into three categories according to their exudate-handling capacity. Foams suitable for low-exudate wounds to be paired with the biosensor for infection detection include Hydrofera and Aquacel. Foams suitable for medium-exudate wounds include Medline Optifoam Basic and Medline Quick. Finally, the foam product best paired with the biosensor for high-exudate wounds is Curafoam.

After bench-testing the compatibility of the biosensors with the foams, two versions of bacterial tests were conducted. In these tests, the biosensors were placed on top of the foams and covered with a transparent adhesive film (i.e., Tegaderm®) to simulate clinical conditions more accurately. In the first bacterial test, no stimulated wound exudate was used, and the foams were placed on top of agar plates (1.0E+5.0 CFU/cm²). Since the foams were not in direct contact with the agar and there was insufficient fluid to transport citric acid from the sensor to the agar, no inhibition zones were observed around the samples. This also delayed exposure of the dye to the lipase.

Referring to FIG. 11C, a biosensor without foam was used as a control to evaluate the color change of foam-incorporated biosensors. As shown, among the tested foams, only Curafoam showed a noticeable color change within the first 6 hours for all three bacterial strains (˜2.0E+6.0 CFU/cm²). For E. coli, additional foams, such as Aquacel, Hydrofera, and Medline Optifoam Basic, began to turn green at the edges, though the color change for P. aeruginosa and MRSA was not as prominent. None of the other foam-paired samples exhibited a color change within the initial 6-hour period. After overnight incubation, other samples without a waterproof membrane, including Medline Quick and those that had already begun to change color, turned dark green. Meanwhile, the control, along with HealQU, Medline Optifoam Non-Adhesive, and Polymem, remained yellow. These results aligned with the bench test findings, confirming that the waterproof layer interferes with the biosensor's sensitivity and overall performance.

Referring to FIG. 11D, color changing responses are shown for biosensors of the present invention paired with different foam on agar with SWF in a bacterial test. SWF was used to replicate the presence of exudate in a wound. Curved agar plates were prepared to ensure the foam-paired biosensor was in direct contact not only with the wound's surface but also with its edges, better mimicking the wound environment. In this setup, the presence of fluid increased the exposure of lipase to the dye, enhancing the interaction between the two. As a result, the color change occurred faster for all samples, as shown. All foams without a waterproof layer, including Aquacel, Curafoam, Hydrofera, Medline Optifoam Basic, and Medline Quick, exhibited a color change for all three bacterial strains tested (3-6 hours). Given that the amount of SWF added at each interval (50 μL) and in total (500 μL) was below the threshold for all samples, no false positives were expected. However, HealQU and Polymem foams did not show a color change due to insufficient fluid penetration. Although a green color change was noticeable in all foams, the color change was less distinguishable with Hydrofera because of its blue color. Overall, the results from these tests suggest that pairing the biosensor with foams that facilitate water penetration from the wound to the biosensor or absorb sufficient wound fluid to expose the biosensor to lipase is effective. The biosensor is compatible with these types of foam dressings.

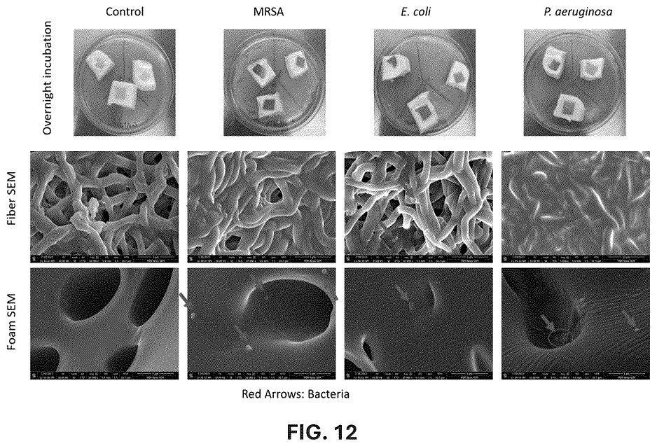

Referring to FIG. 12, it shows the plates after overnight incubation with Curafoam®-paired biosensors and the SEM images of the foam and the biosensors to show the presence of bacteria. To confirm that the color change observed in the bacterial test without SWF was due to lipase activity, the foams and biosensors were fixed and analyzed using SEM to evaluate the presence of bacteria. As shown, even in the absence of SWF, bacteria were able to migrate through the foam and reach the biosensor. Therefore, the color change resulted from lipase secretion by these bacteria, as well as from lipase transferred from the agar to the biosensor via the same pathway. This result confirms that the color change was solely due to the presence of bacteria, with no false positives, as the control samples remained yellow.

Compatibility of the Biosensor with a Wound-Cleaning Solution

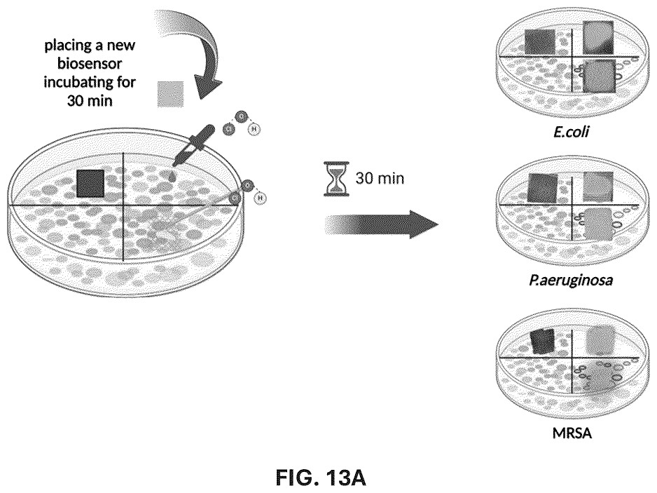

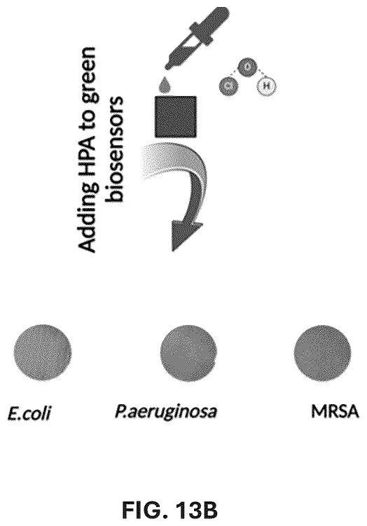

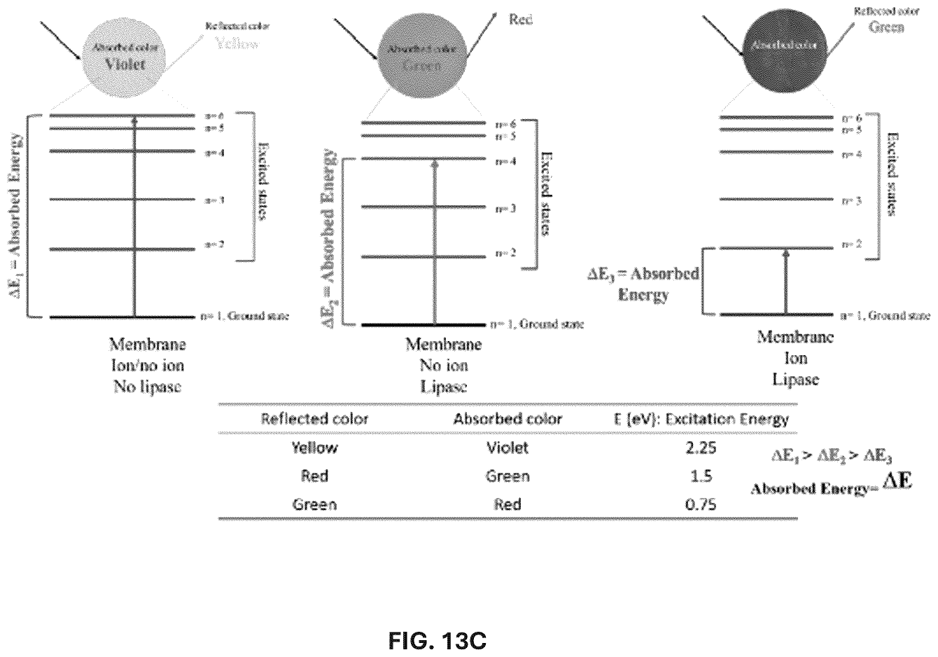

Referring to FIG. 13A, it shows treating the bacteria agar plates with HPA wound cleaning solution either directly pouring the solution or rubbing the agar with gauze soaked in the solution for three different strains of bacteria to evaluate the color change of the biosensors after treatment. In clinical practice, wound washing is a standard procedure, with one of the primary solutions for this purpose being HPA. Therefore, evaluating the performance of biosensors in the presence of this cleaning solution is crucial. To achieve high bacterial concentrations, plates incubated overnight with biosensors were used. The following day, 1 mL of HPA was applied to one quadrant of each plate, while another quadrant was cleaned using gauze soaked in HPA. New biosensors were then placed on the treated areas and monitored for color change within the first 30 minutes, as shown. After the color change in each quadrant, a biopsy punch was taken to measure the bacterial concentration after cleaning treatment to find out the new bacteria load. The control quadrants for all three bacterial strains had concentrations around 10¹⁴ CFU/cm². Final concentrations of treated sections are presented in Table 1.

TABLE 1
Final concentrations of treated sections after
color change of newly placed biosensors.

| | | | Conc. Direct |
|---|---|---|---|
| Bacteria | Initial Con. | Conc. Gauze treatment | solution treatment |
| E. coli | 2.73E+14 ± 4E+13 | 1.27E+05 ± 1.5E+04 | 2.77E+08 ± 3.0E+07 |
| P. aeruginosa | 2.53E+14 ± 4.0E+13 | 3.4E+06 ± 3.0E+5 | 1.2E+09 ± 1.0E+08 |
| MRSA | 2.67E+14 ± 7.6E+13 | 3.4E+05 ± 2.5E+04 | 2.37E+08 ± 1.5E+07 |

For E. coli and P. aeruginosa samples, the biosensors turned green from the edges within 30 minutes. In both strains, the quadrants cleaned with HPA-soaked gauze exhibited a greater log reduction in bacterial load, resulting in less vivid and slower color changes compared to those treated directly with HPA. The log reductions for E. coli were 9 for the gauze-cleaned quadrant and 6 for the direct solution treatment, while for P. aeruginosa, the reductions were 8 and 5, respectively. These results suggest that using HPA-soaked gauze was more effective at reducing bacterial load, as it combined both mechanical and chemical cleaning. However, in both methods, the biosensors detected the remaining bacteria after treatment. The results for MRSA were different. The biosensor did not change color, but a red halo appeared around it, indicating the release of cleaved dye from the biosensor into the agar. The same pattern was observed in the MRSA samples, with a log reduction of 8 for the gauze-cleaned quadrant and 6 for the direct solution treatment.

Referring to FIG. 13B, it shows immersing the color-changed biosensors in the HPA solution to evaluate the color stability. The biosensors that had changed color overnight due to bacterial exposure for all bacteria strains were immersed in HPA to assess their compatibility with the cleaning solution and evaluate the stability of the color. Because HPA has an acidic pH, immersion caused the biosensor color to shift to orange, a mixture of red and yellow, as shown. This occurred because the acid neutralized the salts' effects on dye aggregation. Both green and orange colorations indicate cleaved dye but in different environments.

Referring to FIG. 13C, it shows color theory regarding orange and green colors of the dye after cleavage in different environments. As discussed previously, the presence of salts influences dye aggregation and affects electron layers, as depicted in FIG. 13C. The results of these tests indicated that both methods-cleaning the wound with the solution before applying the biosensors or applying the biosensors first and then cleaning the wound—are effective; the biosensors remain viable in both conditions. Furthermore, if the observed color differs from yellow (either orange or green), it signals the presence of bacteria.

Performance of Biosensors after Sterilization

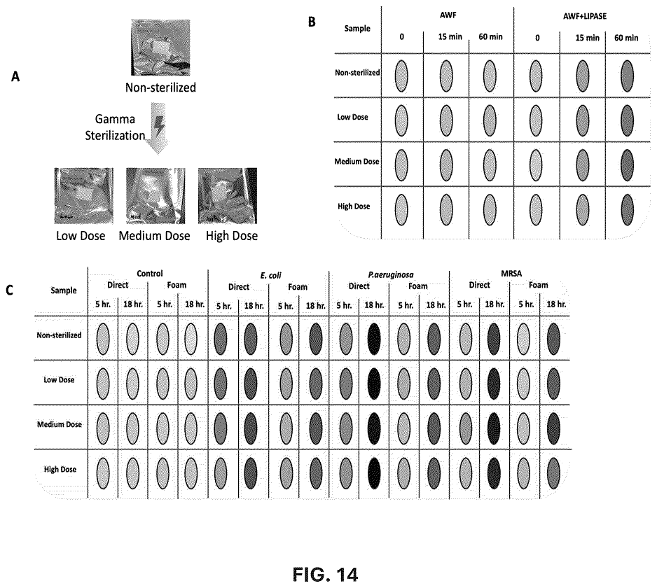

Referring to FIG. 14, it shows A) sterilized samples with three different dosages; B) the results of the bench tests of the sterilized samples; and C) the results of the bacteria tests for sterilized samples. For clinical applications, biosensors must withstand the sterilization process. To test this, the biosensors were exposed to three doses of E-beam radiation (results at 14A). Following sterilization, all samples were subjected to both bench tests and bacterial tests, and their performance was compared to non-sterilized samples to assess whether sterilization impacted detection limits, sensitivity, or accuracy. There were no significant differences in the time required for color change between the sterilized and control samples in the bench test. Additionally, no false positives were observed in any of the sterilized samples. These results indicate that the sterilization process did not deactivate the dye or the citric acid, which is crucial for preventing false positives. In the bacterial tests (E. coli, MRSA, and P. aeruginosa), both direct testing and the use of foams showed that sterilized samples had similar color change timelines as non-sterilized samples, as shown at 14C. This suggests that sterilization did not compromise the sensitivity or accuracy of the biosensors. For all samples, the initial color change was observed within the first 5 hours, with a dark green color developing after overnight incubation. These findings demonstrate that the fabricated biosensors are compatible with sterilization, a critical step in preparing products for clinical use.

### Leaching Test

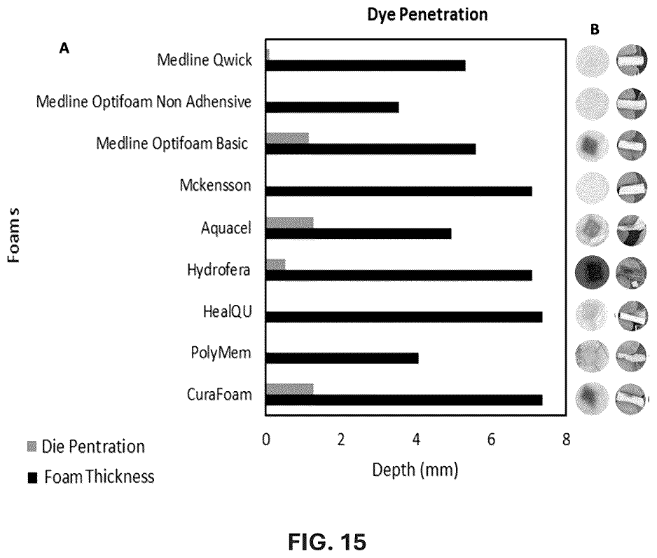

Referring to FIG. 15, it shows A) the foam thickness and penetrated dye through the foam height, and B) the dye stains on top of the foams after removing the biosensors and cross-section of the foams to show the penetrated dye throughout the foams. After drying, the foams and biosensors were removed, and the surface and cross-sections of the foams were examined to detect the presence of dye. As shown as 15A, the dye did not fully penetrate any of the foams, indicating a low risk of dye migration to a wound. As expected, in foams with a waterproof layer (Medline Optifoam Non-Adhesive, Mckesson, HealQU, and Polymem), no dye penetration or cleavage occurred. For Medline Optifoam Non-Adhesive and Mckesson, no dye stains were observed on the surface as shown at 15B. However, in the HealQ and Polymem foams, yellow dye remained on the waterproof layer after the biosensor was removed.

Among the other foams, Medline Qwick showed the least penetration, with 3.5% of the dye penetrating through the foam's height, and no visible dye remained on the top surface. Hydrofera exhibited 9.5% penetration, with the dye residue on the surface forming the complete shape of the biosensor. Medline Optifoam Basic and Curafoam both showed 20% penetration, with the remaining dye matching the shape of the biosensor. Aquacel had the highest penetration, with 30% of the dye reaching the foam's depth, and vivid color remained on the surface due to dye absorption by the foam.

These results indicate that dye penetration from the biosensor to the foam and the surrounding environment is possible. However, the likelihood of dye entering a wound is low, as it does not fully pass through the foams tested-additionally, the residual dye on the foam after biosensor removal aids in visualizing the color change. Since dye penetration does occur, it is essential to assess the toxicity of the released dye.

### Cell Viability

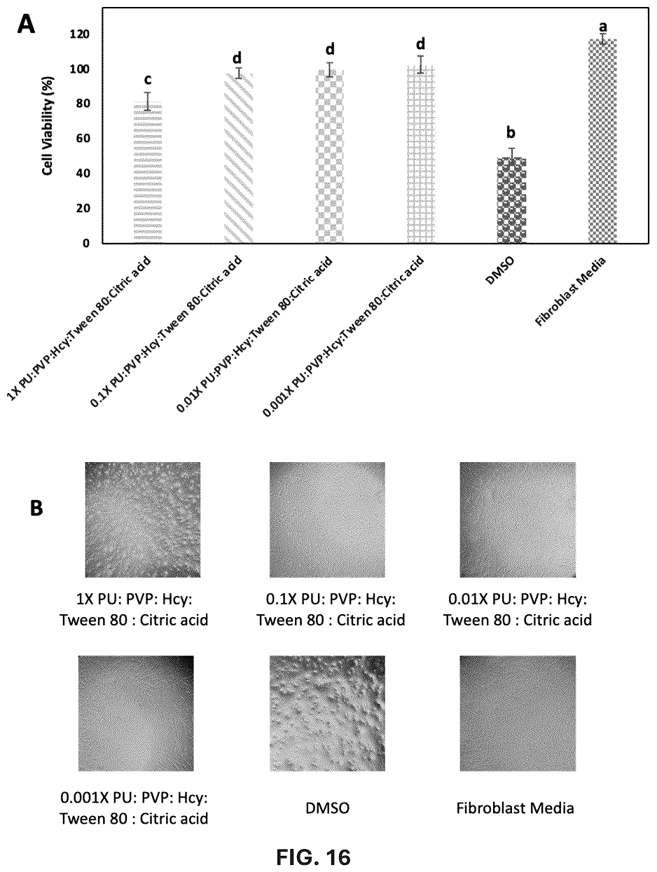

Referring now to FIG. 16, it shows A) the cell viability of Human Adult Skin Fibroblast in the presence of three-day extracted biosensors with different dilutions (1-0.001×) compared to only growth media and DMSO, and B) the morphology cells in the presence of extracts. Fibroblast viability in the presence of the released dye was assessed after three days of extraction. As shown as 16A, biosensors without dilution (1×) exhibited 81.2±5% cell viability, which falls within the acceptable biocompatibility range (80-95%). However, with increasing dilutions-reflecting more realistic wound conditions due to the presence of wound fluids-cell viability significantly improved, reaching 97-100%. The illustration at 16B demonstrates the increase in the population of healthy cells as dilution factors increased. While the number of healthy cells in the undiluted sample was lower than in the control group (fibroblasts in growth media), it was still significantly higher than in the negative control (DMSO). Elongated, healthy cells observed across all diluted samples (0.1×, 0.01×, and 0.001×) confirmed the biocompatibility of both the biosensors and the released dye.

### Theranostic Biosensors Fabrication

ROS-responsive nanoparticles were incorporated into the biosensors of the present invention to develop theranostic biosensors capable of detecting bacteria and signaling their presence to physicians, nurses, and patients via a visible color change while simultaneously releasing antibiotics to prevent infection. These nanoparticles enable triggered antibiotic release in response to bacterial presence, thereby avoiding the sustained release that could lead to bacterial resistance. The following sections present the results of the characterization of the NPs and the nanoparticle-loaded NFs used to fabricate embodiments of the present invention. The ROS-responsive NPs were fabricated using a mPEG₄₅-b-PPS₆₀ polymer, which was synthesized in-house.

### Chemical Characterization of PEG-PPS

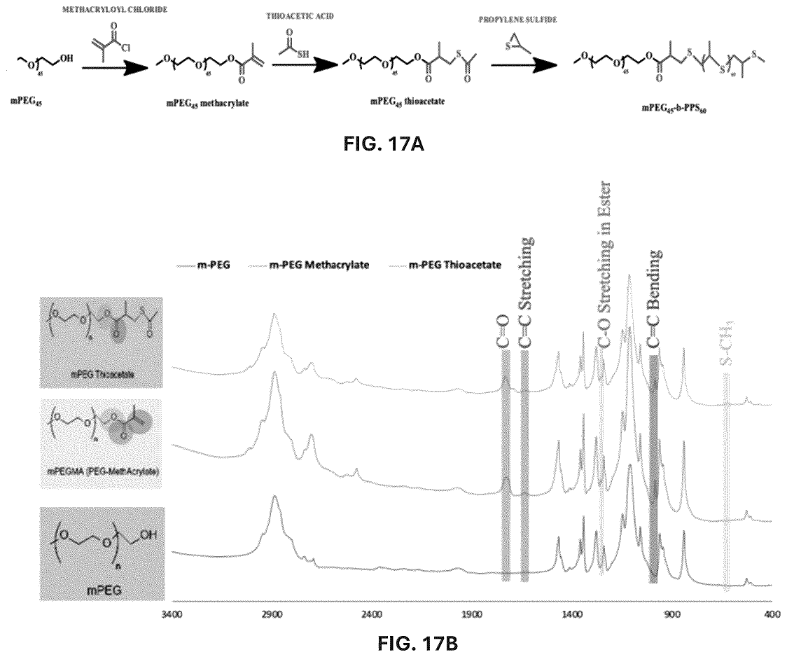

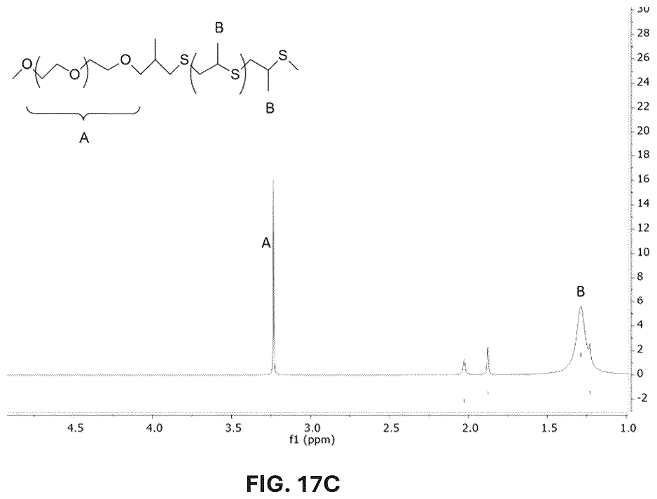

Referring now to FIG. 17A, it shows a schematic of reaction for synthesizing ROS-responsive PEG₄₅-b-PPS₆₀ co-polymer for incorporation into the biosensor of the present invention. The ROS-responsive polymer (mPEG₄₅-b-PPS₆₀) was synthesized in a three-step process, as outlined. First, methoxy poly(ethylene glycol) methacrylate (mPEG₄₅ methacrylate) was synthesized. This was followed by thioacid modification to produce methoxy poly(ethylene glycol) thioacetate (mPEG₄₅ thioacetate), and finally, copolymerization yielded methoxy poly(ethylene glycol)-b-poly(propylene sulfide) (mPEG₄₅-b-PPS₆₀).

Referring to FIG. 17B, it shows FTIR spectra of mPEG, mPEGMA, and mPEG Thioacetate and the NMR spectrum of PEG₄₅-b-PPS₆₀ co-polymer. In the first step, the successful functionalization of m-PEG to PEGMA is demonstrated by the appearance of three new peaks in the FTIR spectrum at 1740 cm⁻¹ (C—O stretching), 1650 cm⁻¹ (C═C stretching) and 980 cm⁻¹ (C═C bending). Additionally, a peak at 1260 cm⁻¹ related to C—O stretching in an ester group further confirms the reaction. This step of synthesizing had a yield of 80%. In the second step, the appearance of a peak at 630 cm⁻¹, attributed to the S—CH₃ group, indicates successful thioacid modification. A reduction in the intensity of peaks associated with the C═C group suggests that some m-PEGMA reacted with thioacetic acid. This step yield was 76.6%.

Referring to FIG. 17C, it shows in NMR data for mPEG45-b-PPS60 a quadruplet peak at 1.81-1.9 ppm (integration 1.93) corresponding to two hydrogens from the O—(CO)HCH₂CH₂S group and a singlet at 2.35 ppm indicating three hydrogens from the —SCOCH₃ group, confirming the successful synthesis of PEG-thioacetate. Additionally, the presence of a peak at 1.35-1.45 ppm in the PEG-PPS spectrum, attributed to CH₃ hydrogens in PPS, further confirms the successful synthesis. The final step had a yield of 50%.

### Fabrication of NPs and Drug Loading

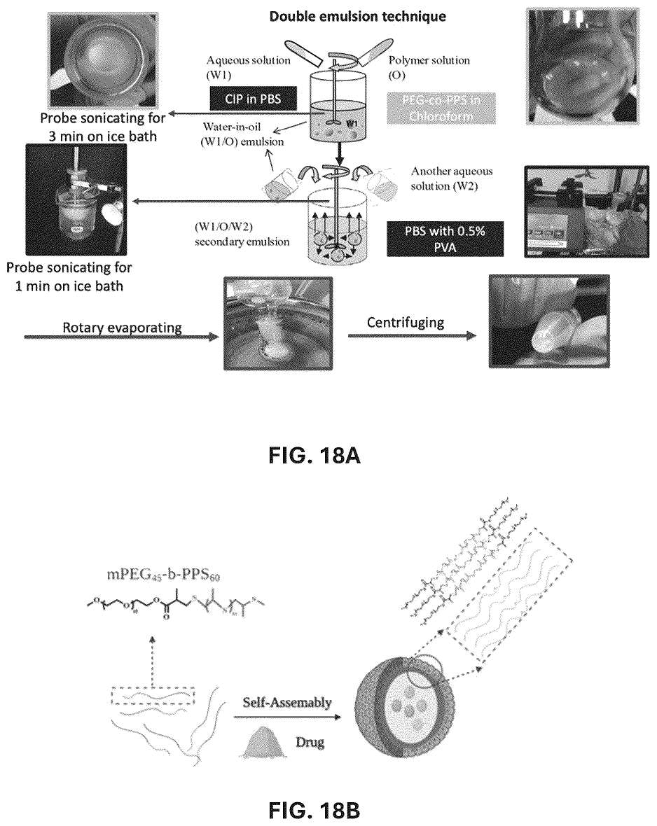

Referring to FIG. 18A, it shows the double-emulsion technique for fabricating ROS-responsive NPs loaded with ciprofloxacin. After successfully synthesizing mPEG₄₅-b-PPS₆₀, NPs were fabricated using a double emulsion technique, as shown. The hydrophilic portion of the polymer, PEG, an FDA-approved material, enhances the serum half-life of the NPs and reduces protein adsorption due to its shielding effect. The final nanostructure morphology depends on the fraction of the PEG component: micelles form when the PEG fraction exceeds 45%, inverted structures form below 25%, and filaments form between 25% and 45%. In preferred embodiments of the present invention, micelles were chosen as the desired structure. Micelles are spherical aggregates of amphipathic molecules (like surfactants or lipids) with a hydrophobic core and a hydrophilic outer shell. This structure allows them to dissolve non-polar substances in water. The dense PEG-based outer layer of these NPs imparts a neutral charge, making them non-immunogenic and non-inflammatory. This outer layer also prevents nonspecific cellular interactions by regulating protein corona formation.

Referring to FIG. 18B, it shows self-assembly of PGE₄₅-b-PPS₆₀. The hydrophobic portion of the polymer, PPS, is responsible for the self-assembly of the NPs and contributes to their stability. PPS is reactive to ROS, and under oxidative conditions, it undergoes structural changes that facilitate the release of the encapsulated cargo.

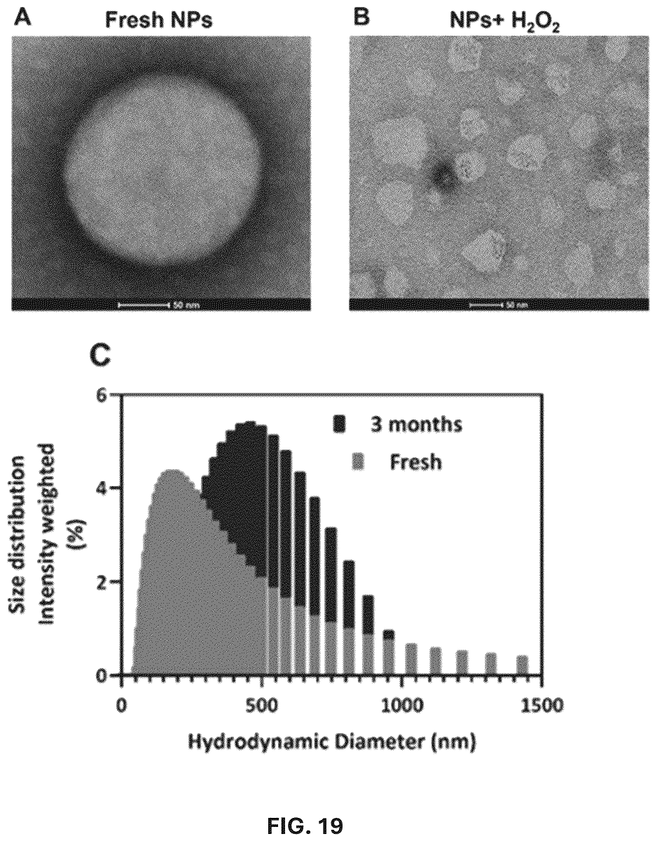

Referring to FIG. 19, it shows at A) a TEM image of freshly fabricated NPs: at B) the TEM image of NPs after treatment with H₂O₂; and at C) the hydrodynamic diameter of fresh NPs and NPs stored in the fridge in PBS for three months. TEM was used to evaluate the morphology of the freshly fabricated NPs, revealing spherical particles with an average diameter of 213.9±29.5 nm, as depicted at A) in FIG. 19. The NPs were fully disassembled after treatment with H₂O₂, and no particles were observed as indicated at B). DLS also indicated a hydrodynamic diameter peak of around 200 nm for fresh NPs. However, when stored in the PBS buffer at 4° C. for three months, the size of the NPs increased to approximately 500 nm, shown at C) in FIG. 19. In contrast, freeze-dried samples maintained consistent size and morphology even after three months of storage.

### Loading NPs in the NF Membranes

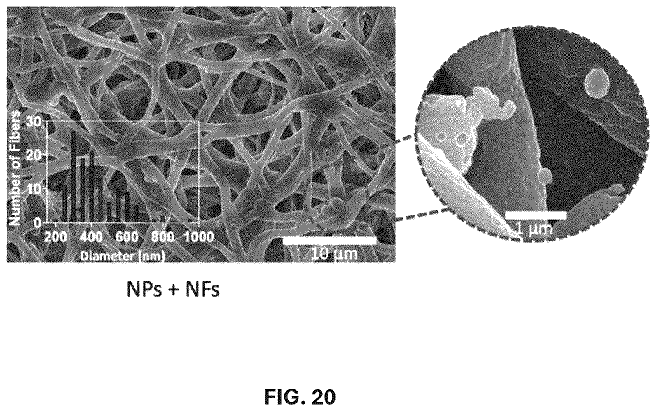

Referring to FIG. 20, it shows the morphology of the theranostic biosensor after immersing in NPs solution and drying. The loading of NPs into NF membranes was achieved by immersing the fabricated biosensors in an NP solution. Consequently, there was a possibility that this process could alter the morphology of both NFs and NPs. To assess this, SEM was conducted after loading and drying the theranostic biosensors. As shown, the distribution and average size of the nanofibers (435±17 nm) remained unchanged, with no significant difference (p-value=0.27) to biosensors without treatment. The SEM images also revealed that the NPs were dispersed between the NFs, and their size showed no significant variation compared to freshly prepared NPs.

Drug Loading Efficiency and Drug Release Behavior from NPs

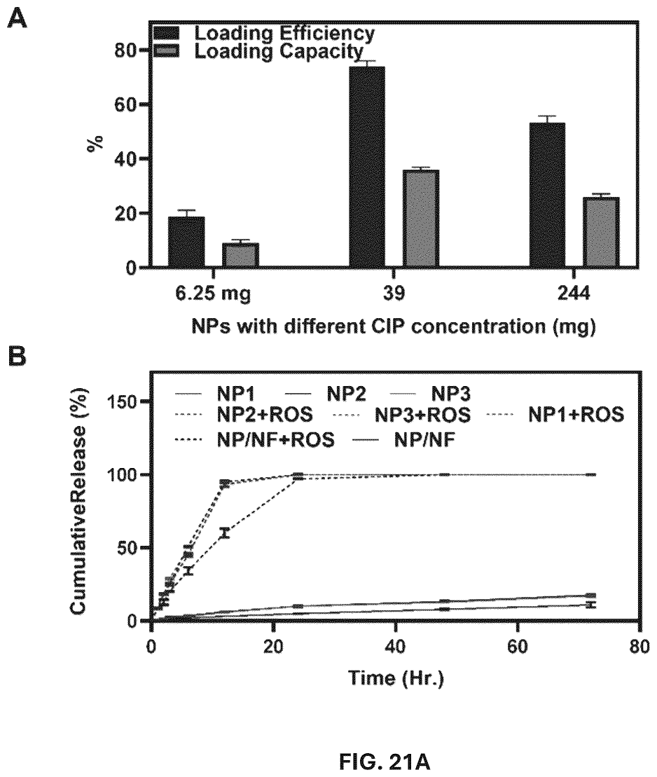

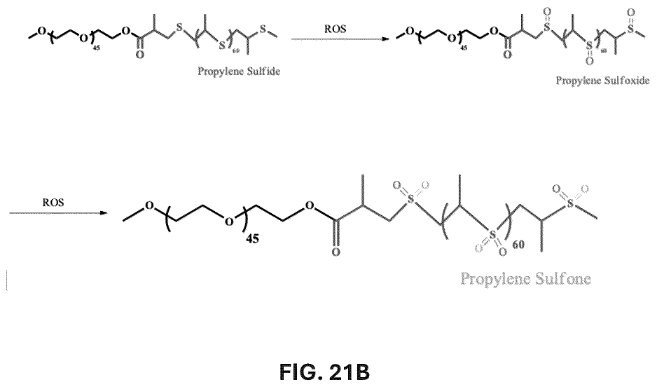

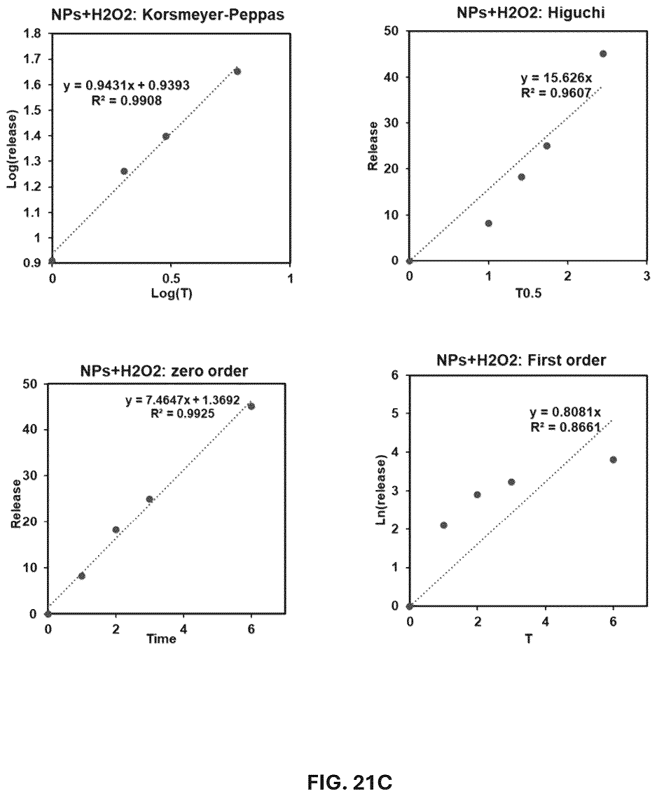

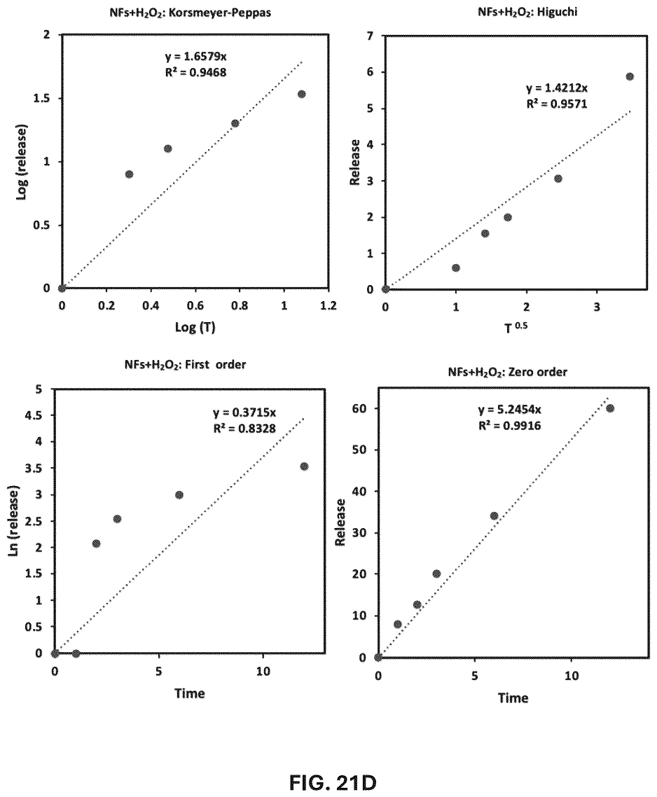

Referring to FIG. 21A, it shows A) the loading efficiency and loading capacity of different concentrations of CIP loaded into NPs, and B) the release profile of CIP from different NPs and NFs loaded with NPs ND. Since three different concentrations of CIP were selected for loading into the NPs, the LE and LC were calculated to determine the optimal concentration for incorporating NPs into the NFs. As shown, the lowest concentration (6.25 mg) resulted in both LE and LC values below 20%. At the highest concentration (244 mg), the LC reached approximately 25%, and the LE was around 53%. Although these values are higher than those observed for the 6.25 mg sample, they are significantly lower than those for the NPs loaded with 39 mg of CIP, which exhibited LE and LC values of 73% and 36%, respectively. The difference between the 39 mg and 244 mg samples may be attributed to the excess drug at higher concentrations, which could lead to saturation of the NP matrix. There appears to be a limit to how much drug can be incorporated into the NPs, and exceeding this limit results in reduced LE and LC. Only drugs with optimal interactions within the NP network remain, while the excess drug is removed during the washing steps in the fabrication process.

The release of CIP from all three NP formulations, as well as from NPs loaded in the biosensor (NP/NF), was evaluated in the absence and presence of hydrogen peroxide (H₂O₂) as a ROS agent. In the absence of H₂O₂, less than 20% of the drug was released from the NPs over 72 hours, and this amount was even lower for the NP/NF samples. This is likely because the drug released from the NPs became further entrapped between the nanofibers, reducing the amount detected in the media at each time point. Overall, this indicates that drug release took longer when NPs were embedded in the biosensor.

Referring to FIG. 21B, it shows the PPS segment undergoes a transition from hydrophobic to hydrophilic, where the structure of m-PEG₄₄-b-PPS₆₀ changes in the presence of a ROS agent converting into hydrophilic poly(propylene sulfone). When H₂O₂ was introduced into the release medium, the drug release profile changed significantly. The release mechanism from PEG-b-PPS nanoparticles (NPs) is driven by the assembly and disassembly of the polymer chains. During NP fabrication, self-assembly occurs due to the hydrophilic nature of PEG and the hydrophobic nature of PPS. In the presence of ROS, however, the PPS segment undergoes a transition from hydrophobic to hydrophilic, as illustrated in FIG. 21B, where the structure of m-PEG₄₄-b-PPS₆₀ changes in the presence of a ROS agent converting into hydrophilic poly(propylene sulfone).

Referring to FIG. 21C, it shows the release models fitted on the release profile in the presence and absence of ROS agent where disassembly of the nanoparticles triggers the release of the encapsulated drug as illustrated. The structural change leads to the disassembly of the nanoparticles, triggering the release of the encapsulated drug as indicated in FIG. 21C. Although the NP/NF samples required more time, both NPs and NP/NF systems achieved 100% release within 72 hours.

Referring to FIG. 21D, it shows NPs and NP/NF systems achieved 100% release within 72 hours. In the case of NPs alone, more than 50% of the drug was released within 6 hours, while the NP/NF system required 12 hours to reach 60% release illustrated in FIG. 21D. The release behavior for all samples followed a zero-order profile, indicating constant drug release after being triggered by the ROS. These results suggest that the NPs were successfully loaded into the NFs and that drug release occurred only in the presence of stimuli.

### Antibacterial Tests

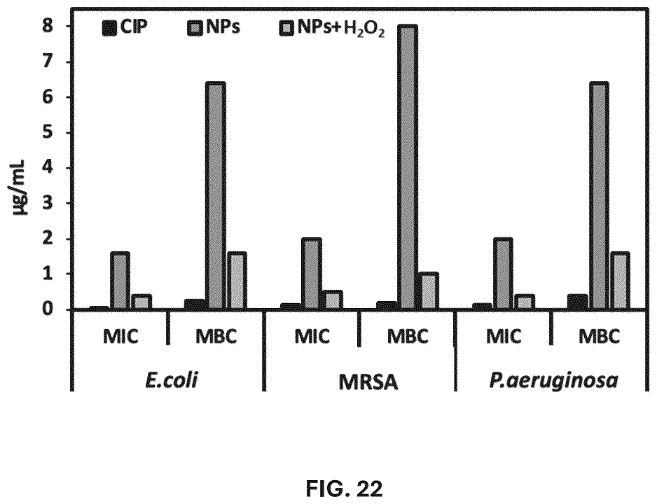

Referring to FIG. 22, it shows MIC and MBC of CIP-Loaded ROS-responsive NPs and CIP in the absence and the presence of H₂O₂ as ROS agent. The Minimum Inhibitory Concentrations (MICs) and Minimum Bactericidal Concentrations (MBCs) of the drug-loaded nanoparticles (NPs) were evaluated against three different bacterial strains—E. coli, MRSA, and P. aeruginosa—both in the absence and presence of H₂O₂ as a ROS agent. These results were compared to CIP as a reference. As shown, the MIC values for the NPs without H₂O₂ were significantly higher for all three bacteria, with MICs being 32, 16, and 16 times greater than those of CIP for E. coli, MRSA, and P. aeruginosa, respectively. However, in the presence of H₂O₂, these differences were notably reduced, with MIC values only 8, 4, and 4 times higher than CIP for the same bacteria.

These findings suggest that CIP loaded into the NPs can be released in response to ROS (H₂O₂), effectively inhibiting bacterial growth at concentrations of 0.4, 0.5, and 0.4 μg/mL for E. coli, MRSA, and P. aeruginosa, respectively. The MBC values followed a similar pattern, with required concentrations for NPs in the presence of H₂O₂ being 1.6, 1, and 1.6 μg/mL for E. coli, MRSA, and P. aeruginosa, respectively, compared to 0.25, 0.2, and 0.4 μg/mL for CIP.

Overall, both the MIC and MBC data demonstrate that the fabricated CIP-loaded NPs possess effective antibacterial activity with acceptable inhibitory and bactericidal properties, particularly in the presence of ROS.

### Zone of Inhibition (ZOI)

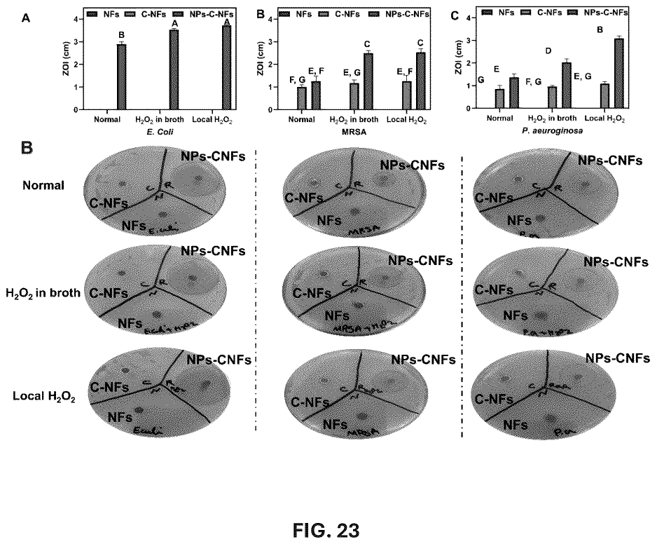

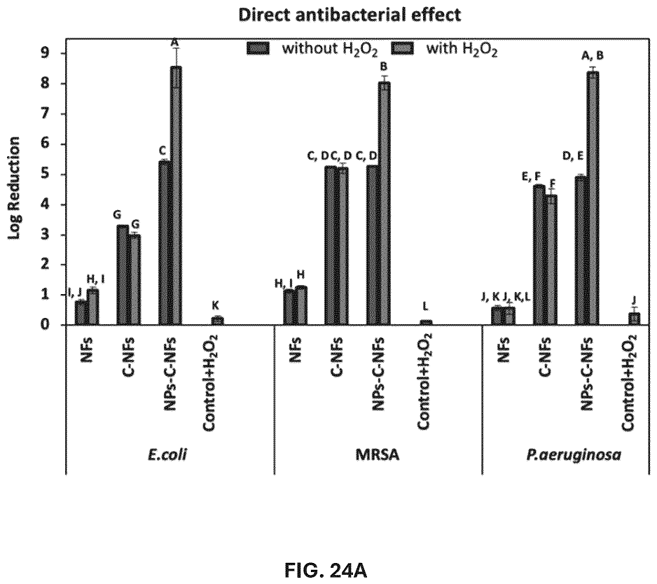

Referring to FIG. 23, it shows A) the Zone of Inhibition (ZOI) data for different bacteria for biosensors (NFs), citric acid-containing biosensors (C-NFs), and theranostic biosensors (NPs-C-NFs) having both citric acid and NPs, where all samples were tested in the absence of H₂O₂ and the presence of H₂O₂ in the broth or locally, and B) the ZOI on agars. To evaluate the antibacterial effect of the theranostic biosensors, a ZOI experiment was conducted under three different conditions: without H₂O₂, with H₂O₂ added to the broth, and with localized immersion of the biosensor in H₂O₂. As shown, the biosensors without NPs or citric acid (NFs) did not show any ZOI for any of the bacterial strains.

The biosensors containing only citric acid (C-NFs) demonstrated an antibacterial effect, showing a ZOI for MRSA and P. aeruginosa but not for E. coli. Interestingly, the addition of H₂O₂ did not significantly alter the ZOI of the citric acid-containing samples. The use of topical acidic solutions to eliminate bacteria, particularly antibiotic-resistant strains, is a well-established practice in wound care management. Acids such as diluted acetic acid, ascorbic acid, and salicylic acid have been used for this purpose. However, due to their potential to harm skin cells, their use has been limited. Citric acid, a natural compound derived from citrus fruits, has emerged as a promising alternative. Its antibacterial mechanism primarily involves altering the pH of the wound environment, making it less suitable for bacterial growth. Additionally, citric acid disrupts bacterial cells by transitioning from its undissociated to dissociated form upon entering the bacteria. This process leads to an internal pH reduction, ultimately causing bacterial cell death. A low intracellular pH damages bacterial DNA, proteins, and cell membranes across various bacterial strains. It has also been proposed that organic acids inhibit nicotinamide adenine dinucleotide (NADH) oxidation by lowering the pH and further promoting cell death. Two additional mechanisms have been suggested for citric acid's antibacterial activity: the first involves direct disruption of bacterial outer membranes, where citrate binds to cell wall components, impairing membrane function and leading to cell death. The second mechanism requires citrate's chelating effect, which captures essential divalent ions like Ca²⁺ and Mg²⁺, both of which are critical for bacterial growth.

Several studies have demonstrated the effectiveness of citric acid against MRSA and P. aeruginosa, showing promising results in inhibiting their growth. However, its effect on E. coli is highly concentration- and pH-dependent. While citric acid can inhibit E. coli growth at very low pH levels (around pH 3), it cannot completely eliminate the bacteria. As the pH increases to 4 or 5, citric acid's effectiveness against E. coli decreases significantly. Based on the data obtained, the amount of citric acid present in the biosensors was insufficient to substantially alter the agar's pH, which explains why no ZOI was observed for E. coli.

For the theranostic biosensors containing NPs (CNPs-C-NFs), the ZOI was observed even without ROS due to the presence of citric acid. However, the introduction of H₂O₂ significantly increased the ZOI compared to samples without H₂O₂. For E. coli and MRSA, there was no significant difference in ZOI whether the H₂O₂ was introduced locally or added to the broth. In contrast, for P. aeruginosa, the locally immersed samples exhibited a larger ZOI. Although the citric acid-containing samples without NPs did not produce a ZOI for E. coli, the theranostic biosensors, even without H₂O₂, showed a larger ZOI for E. coli than for the other two bacterial strains. The significant differences in the ZOI of the theranostic biosensors with and without H₂O₂ indicate that the triggered release of the antibacterial agent was successfully achieved.

### Direct Antibacterial Test

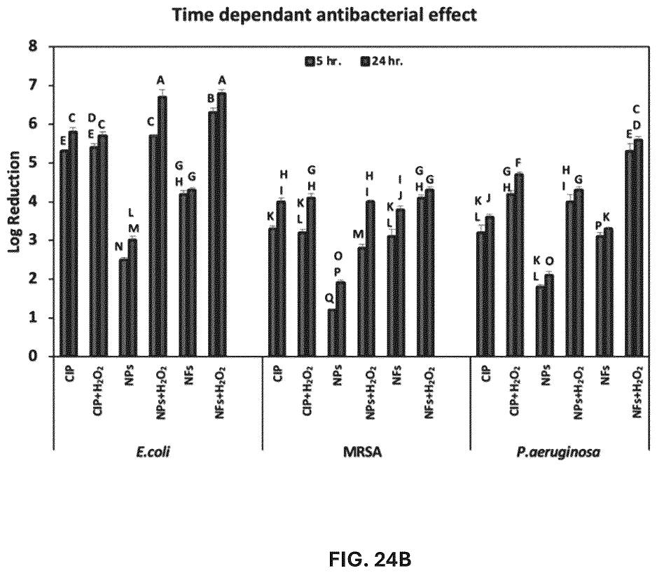

Referring to FIG. 24A, it shows direct antibacterial effect of biosensors with different compositions in the presence and absence of H₂O₂. The antibacterial efficacy of different biosensors—alone (NFs), containing citric acid (C-NFs), and containing both citric acid and nanoparticles (NPs-C-NFs)—was evaluated through direct antibacterial testing to assess the reduction in bacterial counts. As shown in FIG. 24A, the biosensors alone (NFs) achieved less than 1 log reduction in bacterial counts. However, the addition of citric acid to the membrane (C-NFs) resulted in a significant log reduction, with 5 logs for MRSA and 4.6 logs for P. aeruginosa, consistent with the ZOI data. There was no significant difference in these reductions with or without H₂O₂. As previously noted in the ZOI experiments, citric acid had a lesser effect on E. coli, leading to only a 3-log reduction.

In the NPs-C-NFs samples, even without the addition of H₂O₂, the log reduction for E. coli increased significantly to 5.5 logs, aligning with the ZOI data. However, for MRSA and P. aeruginosa, the absence of H₂O₂ did not lead to a significant improvement in log reduction. In contrast, when H₂O₂ was added as a ROS trigger, the log reduction increased dramatically for all bacterial strains, reaching almost 8 logs.

These results demonstrate that the ROS-responsive nanoparticles incorporated into the biosensors can effectively eliminate bacteria in the presence of a ROS agent like H₂O₂, which helps prevent the sustained release of antibiotics and reduces the risk of bacterial resistance. Additionally, in situations where bacterial concentrations are below the infection threshold and the immune system is not actively responding, the citric acid in the biosensors can help control bacterial growth and assist in their elimination even without ROS-triggered release. This dual approach ensures both immediate bacterial control via citric acid and selective antibiotic release in response to elevated ROS levels associated with infection.

### Time Dependent Antibacterial Test

Referring to FIG. 24B, it shows the time-dependent antibacterial effect of CIP, NPs, and NPs-C-NFs in the presence and absence of H₂O₂ for 5 and 24 hours. The continuous antibacterial effect of the theranostic biosensors, as well as NPs and CIP, was evaluated at concentrations below their respective MICs over 24 hours. As shown, except for the NPs-C-NFs in the presence of E. coli, all other samples for all bacterial strains showed a significantly higher log reduction at 24 hours compared to 5 hours. This suggests sustained antibiotic release in the samples exposed to H₂O₂ and the prolonged antibacterial effect of citric acid in the theranostic biosensors, even in the absence of H₂O₂. Furthermore, for all bacterial strains, the NPs without H₂O₂ exhibited a lower log reduction, indicating that minimal untargeted release occurred in the absence of the ROS stimulus.

Among all samples, E. coli showed the highest log reduction, including for CIP, NPs, NPs with H₂O₂, and NPs-C-NFs with H₂O₂, suggesting that this antibiotic is particularly effective against E. coli compared to MRSA and P. aeruginosa. For MRSA, during the initial 5 hours, pure CIP showed a greater log reduction compared to NPs with H₂O₂, indicating that not all the CIP had been released from the NPs. However, in the case of NPs-C-NFs with H₂O₂, the log reduction exceeded that of pure CIP, suggesting that citric acid also contributes synergistically to the antibacterial activity. After 24 hours, the effect of NPs-C-NFs remained greater than NPs alone but similar to that of pure CIP, indicating that more CIP was released over time from the NPs.

For P. aeruginosa, during the first 5 hours, CIP and NPs with H₂O₂ showed similar log reductions, but both were lower than that of NPs-C-NFs, again due to the presence of citric acid. This indicates that the amount of CIP released from the NPs during the initial 5 hours was as effective as pure CIP and more effective than CIP for MRSA. However, after 24 hours, pure CIP achieved a higher log reduction.

Overall, these findings indicate that loading CIP into ROS-responsive NPs did not significantly reduce the antibiotic's effectiveness against bacteria. Additionally, the combination of CIP with citric acid enhanced the antibacterial properties of the biosensors, leading to a higher likelihood of bacterial elimination, even at lower bacterial concentrations and earlier time points.

### Cytocompability

The Cytocompability of the fabricated NPs and NPs-loaded theranostic biosensors were evaluated for human adult fibroblasts to ensure that the synthetic polymer is not cytotoxic.

IC50 for Cip, NPs with and without H₂O₂

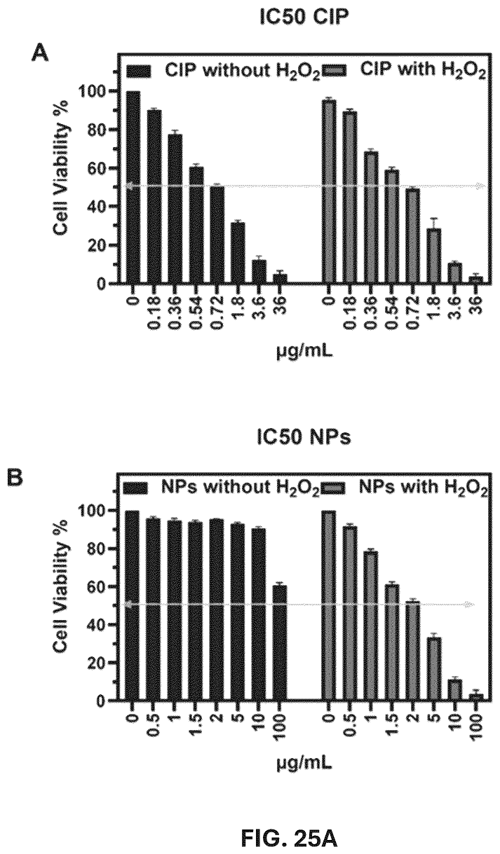

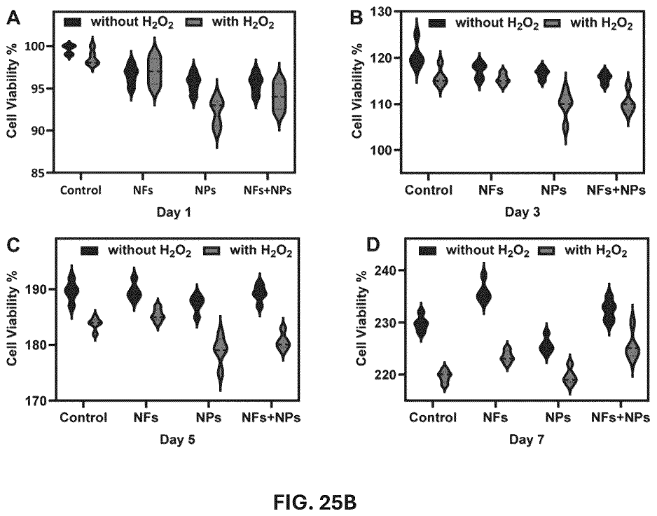

Referring to FIG. 25A it shows A) IC50 of pure CIP in the absence and presence of H₂O₂, B) IC50 of CIP-loaded NPs in the absence and presence of H₂O₂. The IC50 of CIP and CIP-loaded NPs was evaluated in both the absence and presence of H₂O₂, ensuring that the amount of CIP loaded into the biosensor was consistent across all tests. As shown in FIG. 25A, there were no significant differences in cell viability between the presence and absence of H₂O₂ at each CIP concentration, with the IC50 for pure CIP calculated at 0.72 μg/mL.

Referring to FIG. 25B, it shows the Cell viability in the presence of different biosensor extracts at different time points. For the NPs, the concentrations were selected to match the available CIP at each level tested for pure CIP. As indicate, in the absence of H₂O₂, the cell viability for the NPs remained above 80%, even at concentrations as high as 10 μg/mL. A notable decrease in viability, to around 60%, was only observed when the concentration was increased tenfold. This demonstrates that the fabricated NPs are cytocompatible and effectively prevent CIP release, resulting in higher cell viability compared to pure CIP. However, upon the addition of H₂O₂, which triggered the release of CIP from the NPs, cell viability began to decrease as the concentration of available CIP increased.

For the ROS-responsive NPs in the presence of H₂O₂, the IC50 for CIP-loaded NPs was determined to be 2 μg/mL, which corresponds to the same level of effectiveness as 0.72 μg/mL of pure CIP, based on LC. Both the IC50 values for pure CIP and CIP-loaded NPs are above the concentrations used in this project, indicating that cytotoxicity is not a concern for the doses applied in the experiment.

### Cytocompability of NP-Loaded NFs

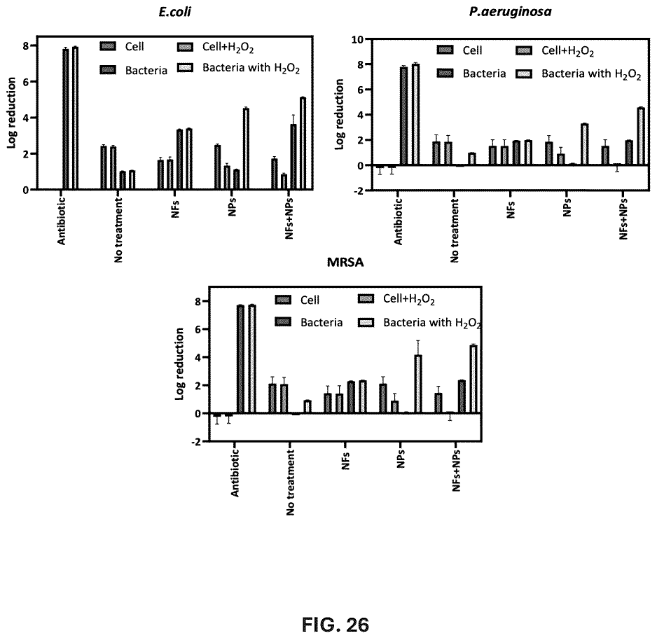

Referring to FIG. 26, it shows log reduction in the number of bacteria and fibroblasts in different conditions in the presence and absence of H₂O₂: cocktail of antibiotics, No treatment, NFs, NPs, and NFs+NPs. A three-day extract from the biosensor (without NPs), NPs, and theranostic biosensor (NFs+NPs) was used to assess the cytocompatibility of the fabricated biosensors in both the presence and absence of H₂O₂. As shown, the presence of H₂O₂ did not result in a significant decrease in cell viability. Over the course of a week, the viability of cells exposed to NPs and NFs+NPs was slightly lower than that of NFs alone and the control group, likely due to the untargeted release of CIP that occurred during the three-day extraction. Despite this slight reduction (5-10%) in viability compared to the control, the theranostic biosensor maintained approximately 90% cell viability.

In terms of cell proliferation, there were no significant differences in the growth rates of cells exposed to NPs, NFs, NFs+NPs, and the control over the one-week period, whether H₂O₂ was present or not. Since the release data indicated that the entire drug load would have been released during the three-day extraction, the one-week cytocompatibility tests with these extracts suggest that the theranostic biosensors are non-cytotoxic, even after complete drug release. Additionally, the lack of any observed changes in cell proliferation rates compared to the control indicates that the biosensor does not impede fibroblast growth, further supporting its biocompatibility.

### A Mixture of Bacteria and Cells

The cell-bacteria co-culturing experiment was conducted to evaluate the antibacterial properties of the theranostic biosensor in the presence of fibroblasts. The results, shown in FIG. 26, are presented in terms of log reduction for both bacterial and fibroblast cells. In the controls treated with antibiotics, all three bacterial strains (MRSA, P. aeruginosa, and E. coli) showed complete bacterial elimination (8 log reduction), and no reduction in fibroblast viability was observed, indicating that the antibiotics eradicated the bacteria while maintaining cell viability. In contrast, the untreated negative controls exhibited almost no log reduction for normal bacteria and less than 1 log reduction for bacteria exposed to H₂O₂. However, the log reduction for fibroblasts was 2, 2, and 3 for MRSA, P. aeruginosa, and E. coli, respectively, indicating that the presence of bacteria led to significant cell death.

For biosensors without NPs, the presence of citric acid increased bacterial log reduction compared to the untreated samples, and fibroblast log reduction decreased to 1 for both MRSA and P. aeruginosa. These results suggest that the citric acid-loaded biosensors had some antibacterial effect, although not complete.

In the NP-loaded samples with H₂O₂, bacterial log reduction was significantly higher than in the NFs-only samples (with H₂O₂), and fibroblast log reduction was significantly lower, indicating that the release of CIP from the NPs effectively reduced bacterial load, allowing more fibroblasts to remain viable. The combined effect of NPs and citric acid was even more pronounced in the NFs+NPs samples. For instance, in the case of E. coli, bacterial log reduction was 1.13 and 1.5 times higher for NFs+NPs than for NPs and NFs alone, respectively. For P. aeruginosa and MRSA, the log reduction was 2.3 and 1.2 times higher than for NFs alone and 1.4 and 2.13 times higher than for NPs alone, respectively.

In the presence of NFs+NPs with H₂O₂, fibroblast viability improved due to the significant reduction in bacterial load. For all bacterial strains, fibroblast log reduction was less than 1, and in the case of MRSA and P. aeruginosa, fibroblasts even began to proliferate, similar to the positive control. These data demonstrate that the theranostic biosensors effectively combat bacterial infections while preserving fibroblast health, making them a promising tool for managing wound infections.

The present invention provides theranostic dressings, particularly those with colorimetric signaling capabilities, which is of critical importance for advancing wound care. The smart dressings of the present invention not only provide real-time feedback on infection status but also enable timely intervention by signaling the presence of bacteria through visible color changes. This feature can assist healthcare providers, patients, and caregivers in monitoring wounds more effectively, reducing the need for invasive diagnostics or frequent dressing changes. In addition, the ability to combine diagnostic functions with therapeutic actions, such as the targeted release of antibiotics, enhances treatment efficiency and reduces the likelihood of prolonged infections or the development of antibiotic resistance. A theranostic dressing that offers both detection and treatment can significantly improve patient outcomes by ensuring timely and appropriate care.

The present invention provides a theranostic biosensor designed to detect bacterial infections, signal their presence via a visible color change, and release antibiotics in a controlled, ROS-responsive manner. Test results demonstrate the present invention's potential for use in wound care, combining effective antibacterial action with biocompatibility.

The nanofibrous biosensors of the present invention including a hemicyanine dye as a probe exhibit highly effective, color-changing properties, providing a clear and reliable visual signal in response to bacterial presence. The biosensor has demonstrated excellent compatibility with different wound dressing foams and cleaning solutions, maintaining its colorimetric response without interference. Additionally, its performance remained accurate under various conditions, such as the presence of salts and proteins typically found in wound environments. Importantly, testing indicated no false positives, even in the presence of these complex biological materials, ensuring the biosensor's reliability in detecting infections and its robustness for practical clinical applications.

For the therapeutic part of the theranostic biosensors of the present invention, the ROS-responsive NPs, based on PEG-PPS, exhibits stable encapsulation of CIP and a triggered release mechanism upon exposure to oxidative conditions, such as the presence of H₂O₂. This triggered release was confirmed by significant increases in bacterial log reduction and ZOI when H₂O₂ was introduced, indicating that the system can prevent untargeted drug release and thereby reduce the risk of bacterial resistance. The combined action of citric acid and CIP-loaded NPs further enhanced antibacterial efficacy, as demonstrated by higher log reductions for MRSA, P. aeruginosa, and E. coli compared to the use of either agent alone.

Cytocompatibility tests confirmed that the biosensors, both with and without NPs, maintained fibroblast viability at over 90%, with no significant cytotoxicity observed even after a complete release of CIP over three days. The co-culture experiments demonstrated that the biosensor's antibacterial properties did not compromise fibroblast health, showing significant reductions in bacterial load while allowing fibroblasts to remain viable and, in some cases, proliferate.

Overall, the theranostic biosensor of the present invention offers an effective solution for managing wound infections. It effectively eliminates bacteria through a combination of ROS-triggered antibiotic release and citric acid's pH-altering properties while preserving cell viability. The ability to control antibiotic release in response to ROS not only reduces the risk of prolonged antibiotic exposure and resistance but also ensures targeted treatment in infected areas. The present invention provides real-time feedback on infection status within a wound and enables timely intervention, signaling the presence of bacteria and fungi at critical thresholds through a visible color change of the nanofibers comprising a nanofibrous matrix, while sequentially releasing in response to ROS an antimicrobial agent loaded into nanoparticles that are immobilized in the matrix. This dual-action biosensor of the present invention provides a valuable tool for improving outcomes in wound healing and infection management.

While the preferred embodiments of the invention have been described above, it will be recognized and understood that various modifications may be made therein, and the appended claims are intended to cover all such modifications which may fall within the spirit and scope of the invention.

In the preceding description, for purposes of explanation, numerous details are set forth in order to provide a thorough understanding of the embodiments. However, it will be apparent to one skilled in the art that these specific details are not required. For example, specific details are not provided as to whether the embodiments described herein are implemented using computer hardware or software, or a combination thereof.

The above-described embodiments are intended to be examples only. Alterations, modifications and variations can be affected to the particular embodiments by those of skill in the art. The scope of the claims should not be limited by the particular embodiments set forth herein but should be construed in a manner consistent with the specification as a whole.

The following references are incorporated herein in their entirety:
1. Chen, G., Deng, H., Song, X., Lu, M., Zhao, L., Xia, S., et al. (2017). Reactive oxygen species-responsive polymeric nanoparticles for alleviating sepsis-induced acute liver injury in mice. Biomaterials, 144, 30-41. https://doi.org/10.1016/j.biomaterials.2017.08.008
2. Liang, J., & Liu, B. (2016). ROS-responsive drug delivery systems. Bioengineering & Translational Medicine, 1, 239-251. https://doi.org/10.1002/btm2.10014
3. Tao, W., & He, Z. (2018). ROS-responsive drug delivery systems for biomedical applications. Asian Journal of Pharmaceutical Sciences, 13, 101-112. https://doi.org/10.1016/j.ajps.2017.11.002
4. Ran, P., Xia, T., Zheng, H., Lei, F., Zhang, Z., Wei, J., et al. (2023). Light-triggered theranostic hydrogels for real-time imaging and on-demand photodynamic therapy of skin abscesses. Acta Biomaterialia, 155, 292-303. https://doi.org/10.1016/j.actbio.2022.11.039
5. Yang, J., He, Y., Li, Z., Yang, X., Gao, Y., Chen, M., et al. (2024). Intelligent wound dressing for simultaneous in situ detection and elimination of pathogenic bacteria. Acta Biomaterialia, 174, 177-190. https://doi.org/10.1016/j.actbio.2023.11.045
6. Pang, Q., Lou, D., Li, S., Wang, G., Qiao, B., Dong, S., et al. (2020). Smart flexible electronics-integrated wound dressing for real-time monitoring and on-demand treatment of infected wounds. Advanced Science, 7. https://doi.org/10.1002/advs.201902673
7. Singh, H., Li, W., Kazemian, M. R., Yang, R., Yang, C., Logsetty, S., & Liu, S. (2019). Lipase-responsive electrospun theranostic wound dressing for simultaneous recognition and treatment of wound infection. ACS Applied Bio Materials, 2(5), 2028-2036. https://doi.org/10.1021/acsabm.9b00076

1. A theranostic wound dressing, comprising:
organic acid and a hemicyanine dye immobilized within polymer nanofibers formed as a nanofibrous matrix:
nanoparticles immobilized within said nanofibrous matrix, said nanoparticles comprising at least one antimicrobial compound encapsulated within the nanoparticles,
wherein, said nanoparticles further comprise a polyethylene glycol b-polypropylene sulfide polymer (mPEG45-b-PPS60) which changes structure in the presence of reactive oxygen species (ROS) said structural change allowing release of said compound, and
wherein, said hemicyanine dye is adapted to react with a color-change when exposed to pathogenic enzymes above specific concentrations of presence.

2. The theranostic wound dressing of claim 1, wherein said organic acid is selected from any of Citric Acid, Ascorbic Acid (Vitamin C), Hyaluronic Acid, Glycolic Acid, and Malic Acid

3. The theranostic wound dressing of claim 1, wherein ester bonds present in said hemicyanine dye are cleavable by said enzymes which cleaving increases intramolecular charge transfer in the hemicyanine dye and induces the color-change.

4. The theranostic wound dressing of claim 1, wherein said antimicrobial compound includes any of ciprofloxacin, clindamycin, doxycycline, minocycline, trimethoprim-sulfamethoxazole (Bactrim), mupirocin (Bactroban), gentamicin, ceftriaxone or analogs thereof.

5. The theranostic wound dressing of claim 1, wherein said nanoparticles comprise ciprofloxacin in a self-assembled polymer structure.

6. The theranostic wound dressing of claim 5, wherein a hydrophobic portion of said mPEG45-b-PPS60 polymer induces self-assembly of said nanoparticles and contributes to their stability.

7. The theranostic wound dressing of claim 5, wherein said mPEG45-b-PPS60 polymer nanoparticles when freshly assembled exhibit spherical morphology with an average diameter of 213.9±29.5 nm.

8. The theranostic wound dressing of claim 1, wherein said enzymes comprise any of proteases, lipases, hyaluronidase, collagenase, coagulase secreted by pathogenic bacteria.

9. The theranostic wound dressing of claim 1, wherein said enzymes comprise any of pectinases, proteases, lipases, chitinases secreted by pathogenic fungi.

10. The theranostic wound dressing of claim 1, wherein said nanofibers in said nanofibrous matrix comprise citric acid in the range of 3% to 5% and fiber diameters in the range from 200 nm to 800 nm, with an average diameter of 412±140 nm.

11. The theranostic wound dressing of claim 1, wherein said nanofibers fibers in said nanofibrous matrix comprise citric acid in the range of 3.8% to 4.2% and said matrix exhibits a thickness of in the range 380-420 μm.

12. A method of fabricating a theranostic wound dressing, comprising:
immobilizing citric acid and a modified hemicyanine dye within polymer nanofibers formed as a nanofibrous matrix by electrospinning;
immobilizing nanoparticles within said nanofibrous matrix, said nanoparticles comprising mPEG45-b-PPS60 polymer encapsulating at least one antimicrobial compound,
wherein, a hydrophobic portion of said mPEG45-b-PPS60 polymer induces self-assembly of said nanoparticles and contributes to their stability,
wherein, said nanoparticles are structurally responsive to reactive oxygen species (ROS), and
wherein, said hemicyanine dye is adapted to react with a color-change in the presence of pathogenic enzymes.

13. The method of claim 12, wherein said enzymes comprise any of proteases, lipases, hyaluronidase, collagenase, coagulase secreted by pathogenic bacteria.

14. The method of claim 12, wherein said enzymes comprise any of pectinases, proteases, lipases, chitinases secreted by pathogenic fungi.

15. The method of claim 12, wherein said nanofibers fibers in said nanofibrous matrix comprise citric acid in the range of 3% to 5% and a fiber thickness in the range of 350-450 μm.

16. The method of claim 12, wherein said nanofibers fibers in said nanofibrous matrix comprise citric acid in the range of 3.8% to 4.2% and a fiber thickness of approximately 380-420 μm.

17. The method of claim 12, wherein said mPEG45-b-PPS60 polymer nanoparticles when freshly assembled exhibit spherical morphology with an average diameter of 213.9±29.5 nm.

18. The method of claim 12, wherein said nanofibers fibers in said nanofibrous matrix comprise citric acid in the range of 3.8% to 4.2% and a fiber thickness of approximately 380-420 μm, and said mPEG45-b-PPS₆₀ polymer nanoparticles when freshly assembled exhibit spherical morphology with an average diameter of 213.9±29.5 nm.

19. The method of claim 12, wherein the structure of m-PEG44-b-PPS60 is adapted to change in the presence of a ROS agent converting into hydrophilic poly(propylene sulfone).

20. The method of claim 12, wherein the chemical structure of the modified hemicyanine dye is characterized as follows: 1H NMR (CDCl3, 300 MHz) δ 7.65-7.72 (m, 3H), 7.27 (d, J=8.6 Hz, 2H), 7.02 (d, J=16.5 Hz, 1H), 2.62 (t, J=7.6 Hz, 2H), 1.84 (s, 8H), 1.41-1.46 (m, 4H), 0.97 (t, J=7.05 Hz, 3H). 13C NMR (DMSO-d6, 75 MHz): δ 177.5, 175.5, 172.0, 153.8, 146.7, 132.4, 131.3, 123.2, 115.8, 113.1, 112.3, 111.2, 100.1, 55.0, 33.9, 31.0, 25.5, 24.4, 22.3, 14.3. MS (ESI): m/z calcd for C24H23N3O3: 401.1739. Found: 400.1651 [M−1]+. FTIR max (cm-1): 2980-2781 (C—H stretch), 2222 (C N stretch), 1770 (C—O stretch), 1255 (C—O stretch).

21. The method of claim 12, wherein micelles comprise the structure of said theranostic wound dressing providing spherical aggregates of amphipathic molecules with a hydrophobic core and a hydrophilic outer shell comprising a dense PEG-based outer layer imparting a neutral charge, making them non-immunogenic, non-inflammatory, and inhibitory to nonspecific cellular interactions by regulating protein corona formation.

22. A theranostic wound dressing, comprising:
citric acid and a modified hemicyanine dye immobilized within polymer nanofibers comprising polyurethane and polypyrrolidone formed as a nanofibrous matrix by electrospinning;
nanoparticles immobilized within said nanofibrous matrix, said nanoparticles comprising mPEG45-b-PPS60 polymer encapsulating at least one antimicrobial compound,
wherein, said nanofibers fibers in said nanofibrous matrix comprise citric acid in the range of 3.8% to 4.2% and exhibit an average diameter of 412±140 nm,
wherein, said nanofibrous matrix has a thickness in the range of 380-420 μm,
wherein, said mPEG45-b-PPS60 polymer nanoparticles when freshly assembled have a spherical morphology with an average diameter of 213.9±29.5 nm,
wherein, said nanoparticles are structurally responsive to reactive oxygen species (ROS), and
wherein, said hemicyanine dye is adapted to react with a color-change in the presence of pathogenic enzymes.

23. The theranostic wound dressing of claim 22, wherein the chemical structure of the modified hemicyanine dye is characterized as follows: 1H NMR (CDCl3, 300 MHz) δ 7.65-7.72 (m, 3H), 7.27 (d, J=8.6 Hz, 2H), 7.02 (d, J=16.5 Hz, 1H), 2.62 (t, J=7.6 Hz, 2H), 1.84 (s, 8H), 1.41-1.46 (m, 4H), 0.97 (t, J=7.05 Hz, 3H). 13C NMR (DMSO-d6, 75 MHz): δ 177.5, 175.5, 172.0, 153.8, 146.7, 132.4, 131.3, 123.2, 115.8, 113.1, 112.3, 111.2, 100.1, 55.0, 33.9, 31.0, 25.5, 24.4, 22.3, 14.3. MS (ESI): m/z calcd for C24H23N3O3: 401.1739. Found: 400.1651 [M−1]+. FTIR max (cm-1): 2980-2781 (C—H stretch), 2222 (C N stretch), 1770 (C—O stretch), 1255 (C—O stretch).

24. The theranostic wound dressing of claim 22, wherein said antimicrobial compound includes any of ciprofloxacin, clindamycin, doxycycline, minocycline, trimethoprim-sulfamethoxazole (Bactrim), mupirocin (Bactroban), gentamicin, ceftriaxone or analogs thereof.

25. The theranostic wound dressing of claim 22, wherein said nanofibrous matrix is configured to signal the presence of bacteria and fungi at critical thresholds below 10⁶ CFU/cm² of wound surface through a visible color change of the nanofibers comprising said nanofibrous matrix, and respond to ROS by sequentially releasing within 12 hours at least 50% of an antimicrobial agent loaded into said ROS responsive nanoparticles that are immobilized in the matrix.
